(19) United States
(12) Patent Application Publication
(10) Pub. No.: US 2019/0392951 A1
(43) Pub. Date: Dec. 26, 2019

(54) MUTATION PROFILE AND RELATED LABELED GENOMIC COMPONENTS, METHODS AND SYSTEMS

(71) Applicant: CALIFORNIA INSTITUTE OF TECHNOLOGY, Pasadena, CA (US)

(72) Inventors: Siddharth JAIN, PASADENA, CA (US); Bijan H.S. MAZAHERI, PASADENA, CA (US); Netanel RAVIV, CULVER CITY, CA (US); Jehoshua BRUCK, LA CANADA, CA (US)

(21) Appl. No.: 16/447,162

(22) Filed: Jun. 20, 2019

Related U.S. Application Data
(60) Provisional application No. 62/687,434, filed on Jun. 20, 2018.

(51) Int. Cl. G16H 50/20; C12Q 1/6886; G06N 20/00; C12Q 1/6827; G16B 10/00

(52) U.S. Cl. CPC G16H 50/20; C12Q 1/6886; G16B 10/00; C12Q 1/6827; G06N 20/00

(57) ABSTRACT

A mutation profile can be determined for an individual's DNA sequence or sequence segment that provides information about the evolutionary history of the DNA. This mutation profile can then be used with a machine learning classifier trained on other people's mutation profiles to determine probabilities that the individual has certain phenotypes. An example is cancer, where the probabilities of different types of cancer can be provided in a disease risk propensity.

### CROSS REFERENCE TO RELATED APPLICATIONS

The present application claims priority to U.S. Provisional Application No. 62/687,434, entitled “Disease Risk Estimation From Mutation Profile Of The Genome” filed on Jun. 20, 2018 with docket number CIT8025-P, the content of which is incorporated herein by reference in its entirety.

### FIELD

The present disclosure relates to a set of data, and related labeled genomic components, methods and systems. In particular, the present disclosure relates to a mutation profile of an individual and to related labeled genomic component methods and systems to obtain a condition risk propensity and/or to predict occurrence of a condition associated with a genomic factor in the individual.

### BACKGROUND

Various conditions have been identified which are associated to genomic factors. Diseases such as cancer obesity, diabetes, heart disease, and mental illness are known to be affected by sequences in the genome of affected individuals.

Current research aims at identifying and detecting the genomic factors that have an effect on disease risk to provide individuals with an indication of their genetic predisposition for these diseases.

Despite much progress in this field, however, identification of parameters that can provide a reliable and accurate determination of a risk propensity for conditions associated with genomic factors is still challenging.

### SUMMARY

Provided herein is a mutation profile indicative of development and diversification of the genome of an individual in time, and related methods and systems which in several embodiments allow determination of the genetic predisposition of the individuals for a condition associated with genomic factors.

In particular, according to a first aspect, a mutation profile of a cell of an individual is described. The mutation profile comprises:

a set of genome values representing history of repeat regions of at least a portion of the genome in of the individual,

each genome value of the set being numerically characterized by a value indicative of

a first number being representative of an error number (m) of the repeat region, and

a second number being representative of a copy number (d) of the repeat region,

the mutation profile indicative of development and diversification of the genome of the individual in time.

The profile can be stored on a non-transitory computer-readable medium, such as a hard drive, flash drive, or other computer memory storage.

The mutation profile can be described in several mathematically-equivalent ways:
1. As a matrix:

$$( \begin{matrix}{m}_{1} & {m}_{2} & {m}_{3} & … & {m}_{i} \\ {d}_{1} & {d}_{2} & {d}_{3} & … & {d}_{i}\end{matrix} ) .$$

2. As a vector, each entry of whom is an (mᵢ, dᵢ) pair:
   ((m₁, d₁), (m₂, d₂), (m₃, d₃), . . . (mᵢ, dᵢ)).
3. Other variations: Other versions of mutation profile are possible, including those with more than two values per entry (i.e. other than m and d). For example, this could be [(m₁, d₁, l₁), (m₂, d₂, l₂), (m₃, d₃, l₃), . . . ] where lᵢ is the length of the seed. One can also include the seed itself in each entry. As another example, the m values can be split into sub-categories of error (such as separate values for insertion, deletion, and substitution.).
   In the mutation profile, the values (mᵢ, dᵢ) of repeat region i can be determined according to the most probable history of mutations. One other variation is to consider all histories simultaneously, by computing a respective pair (mᵢ^{(j)}, dᵢ^{(j)}) for every possible history j, and defining the values (mᵢ, dᵢ) as a weighted average, or expectation: (mᵢ, dᵢ)=Σⱼ Prᵢ(j)·(mᵢ^{(j)}, dᵢ^{(j)}), where Prᵢ(j) stands for the probability that the true history of the i-th repeat region is the j-th one.

According to a second aspect, a method is described for building a mutation profile indicative of development and diversification of the genome of an individual in time described herein.

The method comprises: finding at least one repeat region in a genomic sequence from the individual; and evaluating a consensus pattern for each of the at least one repeat region.

The method further comprises determining a plurality of mutation histories to each of the at least one repeat region and each mutation history having a consensus pattern; determining estimated histories for each of the plurality of mutation histories for each consensus pattern; and building a mutation profile based on the estimated histories of the plurality of mutation histories for each consensus pattern. The mutation profile can be constructed by associating a mutation index corresponding to the most probable mutation history for each of the at least one repeat regions. In some embodiments, the method can further comprise obtaining a DNA sequence from the individual, for example by sequencing the genome of the individual or a portion thereof to provide the genomic sequence from the individual.

According to a third aspect, a method is described for determining a condition risk propensity for a target condition in an individual. The method comprises: determining a first set of mutation profiles for a population of individuals with the target condition, each mutation profile of the first set of mutation profiles being the mutation profile according to the present disclosure for each corresponding individual of the population of individuals with the target condition.

The method further comprises determining a second set of mutation profiles for a population of individuals not having the target condition, each mutation profile of the second set of mutation profiles being the mutation profile according to the present disclosure for each corresponding individual of the population of individuals not having the target condition.

The method also comprises training a classifier using the first set of mutation profiles and the second set of mutation profiles; and running the classifier on a mutation profile of the individual such that a risk propensity for the target condition is generated from the mutation profile of the individual being the mutation profile described herein for the individual. In some embodiments the target condition is a single condition and the in some embodiment the target condition is a plurality of target conditions the determining the first set of mutation, the determining the second set of mutation and the training is performed sequentially for each condition.

According to a fourth aspect method is described for determining a condition risk propensity for a target condition in an individual. The method comprises: determining a plurality of sets of mutation profiles for a plurality of populations comprising a populating having the target condition, each of the plurality of populations having a corresponding condition unique to that population, each mutation profile being a mutation profile described herein for an individual of the plurality of population. The method further comprises training a classifier using the plurality of sets of mutation profiles, classifying by condition; and running the classifier on a mutation profile of the individual such that a risk propensity is generated for the target condition, the mutation profile of the individual being the mutation profile in accordance with the present disclosure for the individual. In some embodiments the target condition is a single condition and the in some embodiment the target condition is a plurality of target conditions. In some of these embodiments each of the plurality of populations has a unique condition of the target conditions.

According to a fifth aspect, a method is described to predict a condition risk propensity of an occurrence of a target condition in an individual, the target condition associated with genomic factors. The method comprises detecting, in a cell of the individual, a mutation profile described herein, the detected mutation profile indicative of development and diversification of the genome of the individual in time. The method further comprises comparing the detected mutation profile with a reference mutation profile associated with the condition to provide a condition risk propensity for the individual.

According to a sixth aspect. a method is described to identify a distance between different conditions, the method comprising building at least one classifier, wherein a first condition and a second condition are classified by the at least one classifier; determining a classification accuracy for the first condition against the second condition; and determining a condition distance based on the classification accuracy.

The mutation profile and related methods, systems, condition risk propensity and labeled human genome component herein described are based on considering repeat regions of a genome of an individual as a nature-given repetition error-detecting code.

In particular, mutation profile and related methods, systems, condition risk propensity and labeled human genome component herein described the repeat regions and the point mutation errors in the repeat regions are detected and analyzed to detect information about the history of the evolution of these regions, which effectively characterize an evolution channel. Such evolution channel can shed light on the accumulation of mutations in the genome, which is a temporal feature of the genome.

The mutation profile and related methods, systems, condition risk propensity and labeled human genome component herein described allow in several embodiments to quantify in an informative manner evolutionary information of the genome of an individual for the purpose of evaluating a genetic predisposition of an individual for one or more target conditions associated with genomic factor.

The mutation profile and related methods, systems, condition risk propensity and labeled human genome component herein described allow in several embodiments to leverage a link between one or more target condition and genomic sequences inherited from ancestors as well as sequences developed at birth or later during a lifetime of an individual and to develop a test which estimates the individual's inclination to contract the condition.

The mutation profile and related methods, systems, condition risk propensity and labeled human genome component herein described can be used in several embodiments, to provide the first statistical test which obtains non-negligible probability of prediction of a condition associated with genomic factors from genome-wide analysis of healthy cell DNA, and moreover, offers a general approach that can be applied over any genetic condition.

The mutation profile and related methods, systems, condition risk propensity and labeled human genome component herein described can be used in several embodiments, to identify a distance between different type of cancers.

The mutation profile and related methods, systems, condition risk propensity and labeled human genome component herein described allow in several embodiments early screening of individuals for many of conditions, such as various forms of cancer, resulting from an intricate mixture of complex and not well understood factors involved, factors.

The mutation profile and related methods, systems, condition risk propensity and labeled human genome component herein described can be used in connection with various applications wherein information concerning development and diversification of the genome of an individual in time and in particular a mutation profile and/or a condition risk propensity for an individual, are desired. For example, method and systems herein described can be used in clinical application to diagnose a condition in an individual and/or to predict likelihood of occurrence of a condition in an individual. In particular, methods and systems herein described and related profiles can be used in statistical tests conducted to advise individuals at risk to go through examinations earlier and/or more frequently. Additional exemplary applications include basic biology research, applied biology, agriculture, bio-engineering, medical research, medical diagnostics, therapeutics, and additional fields identifiable by a skilled person upon reading of the present disclosure. For example, identification of a mutation profile and/or a condition risk propensity for a target condition can link the mutation activity captured by mutation profile with mutations that are causing cancer.

The details of one or more embodiments of the disclosure are set forth in the accompanying drawings and the description below. Other features, objects, and advantages will be apparent from the description and drawings, and from the claims.

### BRIEF DESCRIPTIONS OF THE DRAWINGS

The accompanying drawings, which are incorporated into and constitute a part of this specification, illustrate one or more embodiments of the present disclosure and, together with the detailed description and example sections, serve to explain the principles and implementations of the disclosure.

FIG. 1 shows a schematic representation of genomic factors and their effect on a condition, such as cancer.

FIG. 2 shows an example of tandem duplication errors in a sequence.

FIG. 3 shows an example of two possible paths of genomic mutation history for a repeat region.

FIG. 4 shows an illustration of the controlling factors for an evolution channel.

FIG. 5A-5F show an example history of non-repeat mutations of different types.

FIG. 6 illustrates examples of data sources for DNA sequences for the methods and systems described.

FIGS. 7A and 7B show examples of a machine learning flow for some embodiments of the described systems and methods of the disclosure.

FIGS. 8A and 8B show an example of accuracies and sensitivity/specificity scores for an embodiment of a classifier for the described systems and methods based on mutation profiles.

FIGS. 9A and 9B show an example of accuracies and sensitivity/specificity scores for a further embodiment of a classifier for the described systems and methods based on ratios of error numbers and copy numbers.

FIGS. 10A and 10B show an example of accuracies and sensitivity/specificity scores for a further embodiment of a classifier for the described systems and methods based on average values of the ratios of error numbers and copy numbers.

FIGS. 11A-11F show examples of average risk profiles for various cancer patients using binary classification.

FIGS. 12A-12F show examples of average risk profiles for various cancer patients when a gradient boosting algorithm is employed.

FIGS. 13A-13F show examples of average risk profiles for various cancer patients when a gradient boosting algorithm is employed with multi-classification.

FIG. 14 shows an example of training and testing of an embodiment of machine learning for the systems and methods described.

FIG. 15 shows a further example of training and testing an embodiment of machine learning for the systems and methods described.

FIGS. 16A and 16B show an example of accuracies and sensitivity/specificity scores for an embodiment of a classifier for the described systems and methods based on mutation profiles from purified samples.

FIG. 17 shows and example of binary classifier accuracies for a risk propensity for leukemia, brain, and ovary cancer.

FIGS. 18A and 18B show an example of 4-fold validation accuracy and sensitivity/specificity, for purified samples, for four main clusters of cancers.

FIGS. 19A and 19B show an example of example mean and standard deviations for the cancer classifications when purified samples are used.

FIG. 20 shows an example of an indexed mutation profile.

FIG. 21 shows an example of using a multi-classifier to show the risk of brain cancer in an individual.

FIG. 22 shows an example of using a multi-classifier to show the risk of skin cancer in an individual.

FIG. 23 shows an example of using a multi-classifier to show the risk of pancreatic cancer in an individual.

### DETAILED DESCRIPTION

Provided herein is a mutation profile indicative of development and diversification of the genome of an individual in time, and related labeled genomic components, methods and systems.

The term “mutation” as used herein indicates an alteration of the nucleotide sequence of a genome, typically resulting from errors during DNA replication (especially during meiosis) or other types of damage to DNA (such as may be caused by exposure to radiation or carcinogens), which then may undergo error-prone repair (such as microhomology-mediated end joining), or cause an error during other forms of repair, or else may cause an error during replication (e.g. translesion synthesis). Mutations can also result from insertion, substitution, or deletion of segments of DNA due to mobile genetic elements of the genome.

The term “genome” as used herein indicates the genetic material of an organism. In particular, a genome indicates genetic material which contains all of the information needed to build and maintain an organism. A genome is formed by nucleic acids which can be detected and/or isolated alone or in combination with other molecules present in the organism.

The term “nucleic acids” “polynucleotides” as used herein refer to an organic polymer composed of two or more monomers including nucleotide or nucleosides or analogs thereof. In particular, the term “polynucleotides” of a genome indicates biological molecules comprising a plurality of nucleotides or nucleosides. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “polynucleotide” includes nucleic acids of any length. Polynucleotides in the sense of the disclosure comprise biological molecules comprising a plurality of nucleotides and/or nucleosides. Polynucleotides can typically be provided in single-stranded form or double-stranded form as will be understood by a person of ordinary skill in the art.

Exemplary nucleic acids include deoxyribonucleic acids (DNA) and ribonucleic acids (RNA), each synthesized from four different types of nucleotides, also called “bases”. The nucleotides for DNA include deoxy-adenosine (“A”), deoxy-thymidine (“T”), deoxy-cytosine (“C”), and deoxy-guanosine (“G”). The nucleotides for RNA include adenosine (“A”), uracil (“U”), cytosine (“C”) and guanosine (“G”). The nucleotides of a DNA or RNA are arranged in a particular order, referred to as the sequence of the DNA or RNA. The order of nucleotides, the four bases, within a DNA or RNA molecule is determined using nucleic acid sequencing methods.

A genome in the sense of the disclosure typically consists of DNA (or RNA in RNA viruses) and includes both the coding DNA (genes) and the noncoding DNA of the genome, as well as mitochondrial DNA and chloroplast DNA of the organism.

The term “nucleotide sequences as used herein indicates a succession of letters that indicate the order of nucleotides within a DNA (using GACT) or RNA (GACU) molecule. By convention, sequences are usually presented from the 5′ end to the 3′ end. For DNA, the sense strand is used. Because nucleic acids are normally linear (unbranched) polymers, specifying the sequence is equivalent to defining the covalent structure of the entire molecule. For this reason, the nucleic acid sequence is also termed the primary structure and the wording is used to indicate both the polynucleotide and the information conveyed by the succession of letters forming the sequence.

A genome sequence indicates the complete list of the nucleotides (A, C, G, and T for DNA genomes) that make up all the chromosomes of an individual or a species. Within a species, the vast majority of nucleotides are identical between individuals, but some nucleotide sequences are unique of specific individuals.

The genome includes both the coding regions (genes) and the noncoding DNA of the organism, as well as the genetic material of the mitochondria, and chloroplasts of an individual. The genome of different organisms in a same or different taxonomic group presents a difference in sequences which is known as genetic variation which is a result of mutations occurring in the individual in time.

The term “individual” “organism” or “subject” as used herein indicates. a single biological multicellular organism. Typically, all individuals are capable of reproduction, growth and development, maintenance, and some degree of response to stimuli. Preferably individuals in the sense of the disclosure refers to plants or animals and in particular higher animals such vertebrates and in particular mammals, such as cow, horses, goats and other livestock, and more particularly human beings.

Mutations in the sense of the disclosure include duplication, insertions, deletion, substitutions as well translocations and inversion of one or more nucleotides of the nucleotide sequences, as well as chromosome cross over as will be understood by a skilled person.

Mutations of the genome may or may not produce discernible changes in the observable characteristics (phenotype) of an individual. Mutations play a part in both normal and abnormal biological processes including evolution, cancer, and the development of the immune system, including junctional diversity.

As a result, various conditions of an individual can be associated to the nucleotide sequence of the individual and its variation over time through mutations of the genome (herein collectively indicated as genomic factors).

The term “condition” indicates a physical status of the body of an individual (as a whole or as one or more of its parts e.g., body systems), that does not conform to a standard physical status associated with a state of complete physical, mental and social well-being for the individual. Conditions herein described comprise disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms in an individual. Conditions in the sense of the disclosure also comprise a physical status developing through aging, and not conform to a standard physical status associated with a state of complete physical, mental and social well-being for the individual. such as baldness or myopia.

The wording “associated to” as used herein with reference to two items indicates a relation between the two items such that the occurrence of a first item is accompanied by the occurrence of the second item, which includes but is not limited to a cause-effect relation and sign/symptoms-disease relation.

Conditions associated to genomic factors include cancer, autism, Crohn's disease, Duchenne muscular dystrophy, hemochromatosis, Huntington's disease, Turner's syndrome, congenital heart diseases, autoimmune diseases, Parkinson s disease, and others identifiable by a skilled person.

In embodiments of the present disclosure repeat regions of the genome of the individual and point mutation errors in the repeat regions are detected and analyzed to provide information about the accumulation of mutations in the genome of an individual in time.

The term “repeat region” or “repeated sequences” (also known as repetitive elements, repeating units or repeats) indicate patterns of nucleic acids that occur in multiple copies throughout the genome. More than 50% of the human genome consists of repeated sequences [1]. Repeat regions can be categorized as tandem repeat regions, interspersed repeat regions, or any other discoverable repeat pattern type in a genome.

Tandem repeats are repeats which lie adjacent to each other on the genome, either directly or inverted. Exemplary tandem repeats comprise satellite (DNA typically found in centromeres and heterochromatin) minisatellite (repeat units typically from about 10 to 60 base pairs, found in many places in the genome, including the centromeres), and microsatellite (repeat units typically less than 10 base pairs; such as telomeres, having 6 to 8 base pair repeat units). Tandem repeats are caused by slipped-strand mispairings [2]. Slipped-strand mispairings occur when one DNA strand in the duplex becomes misaligned with the other.

Interspersed repeats are repeats dispersed throughout the genome and nonadjacent, and comprise transposable elements, (e.g. DNA transposons and retrotransposons such as LTR-retrotransposons (HERVs), and non LTR-retrotransposons). which can copy or cut and paste itself into new positions of the genome.

Repeat regions can also be categorized as direct repeats occurring when a sequence is repeated with the same pattern downstream, and inverted repeats occurring when a single stranded sequence of nucleotides is followed downstream by its reverse complement. Direct repeats can be typically tandem repeats, interspersed repeats or flanking (or terminal) repeats (terminal repeat sequences) repeated on both ends of a certain sequence. Inverted repeats can be found in transposons, palindromes (when there is no intervening sequence) pseudoknots and riboswitches.

Repeated regions of the genome are a source of genetic variation and regulation and genome. Accordingly in embodiments of the present disclosure tandem repeats and other repeat regions and point mutations of the repeat are detected and analyzed to provide information on the evolution of the genome, a temporal feature of the genome which effectively characterize an evolution channel contributing to the occurrence of the condition of the individual.

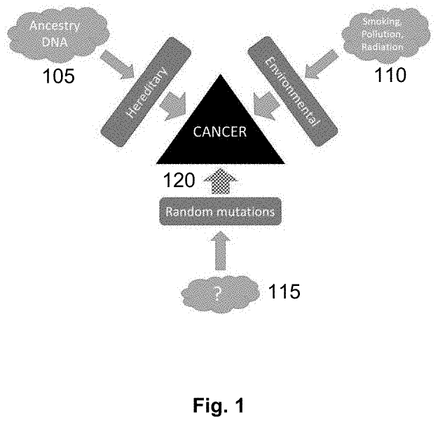

FIG. 1 shows a schematic illustration of the various factors in the evolution channel, contributing to genetic disease like cancer. Unlike Hereditary (105) and Environmental (110) mutations, Random mutations (115) occur in every individual. However, the cause of cancerous random mutations is unknown. An embodiment of the present systems and methods can identify which types of random mutations are the ones which correlate with cancer, namely, to analyze the effect of the arrow (120).

Random mutations occur naturally during DNA replication in stem cell divisions. DNA Replication might trigger slipped stranded mispairing as a result of slippage, creating repeat regions throughout the genome. These repeat regions, known as microsatellites when the length of the repeated pattern is less than 10 (due to current technological limitations), are often imperfect due to substitutions, insertions and deletions. Further, the mutation of these regions is known to increase with age and is not known to be linked to be any particular gene. A recent study [3] has demonstrated a striking correlation between the number of stem cell divisions and incidence of cancer, a finding that strongly encourages the assumption that random mutations are to blame for in many cases.

Even prior to [3], numerous studies have addressed the consequences of repeat regions [4], [5], [6], [7]. However, all previous studies characterized a repeat region by a one-dimensional approach that only considers the number of repeats or their length, and the rich body of knowledge that can be extracted from a repeat region was largely ignored.

In embodiments herein described, mutational events are detected by analyzing various properties of the repeat regions in cells using the structure of repeats to infer information about the mutation accumulation process).

The term “cell” as used herein indicates the basic structural, functional, and biological unit of all known living organisms. All cells, have a membrane that envelops the cell, regulates what moves in and out (selectively permeable), and maintains the electric potential of the cell. Cells typically comprise DNA, the hereditary material of genes, and RNA, containing the information necessary to build various proteins such as enzymes, the cell's primary machinery. There are also other kinds of biomolecules in cells.

Cell of an individual can be organized in tissues wherein a “tissue” is a cellular organizational level between cells and a complete organ. In particular A tissue is an ensemble of similar cells and their extracellular matrix from the same origin that together carry out a specific function. Organs are then formed by the functional grouping together of multiple tissues.

In some embodiments, repeats analyzed with methods and systems of the disclosure are obtained from a healthy cell of the individual according to an approach that was not explored in the past. The term “healthy” when referred to cells, DNA, sequences, tissues and additional information or material of an individual indicates a reference item not displaying signs of a condition and in particular, of the target condition. For example, if the condition is cancer, then the cancerous cells and/or tissues are “unhealthy” and the non-cancerous cells are “healthy”. By “mutation profile of a cell”, it can be either a healthy cell or unhealthy cell, and for most practical purposes by current technology, will be determined from a sample of many cells drawn from a tissue and/or common location (e.g. a blood sample), so “a cell” includes a plurality of cells taken as a group.

Measurements of repeats from a cell of an individual can be conducted from multiple tests along a person's life, but the present systems and methods outline a one-shot approach providing information about the evolution of the genome of the individual.

In embodiments of mutational profiles, and related labeled genomic components methods and systems, herein described, the mutational events characterizing this evolution channel can be divided into two categories: duplications which result in repeat regions and point mutations. While evolution through point mutations is unconstrained, giving rise to exponentially many possibilities of what could have happened in the past, evolution through duplications adds constraints limiting the number of those possibilities.

In particular mutational profiles, and related labeled genomic components methods and systems, herein described are based on the observation genome has evolved through a series of mutational events spanning generations, giving rise to tremendous diversity between individuals. Each individual's genome is a realization of a distinct evolution channel, which is a function of hereditary, environmental, and stochastic factors. By observing an individual's genome one can see the effects of this underlying evolution channel. including propensity of the individual to develop a condition.

Mutation profiles and related labeled genomic components, methods and systems herein described comprise genome values that represent history of repeat regions of at least a portion of the genome of the individual.

Since there are several repeated regions in DNA, one can aggregate this evolutionary information of each repeated region and use it as a model that can provide information about the evolution of genome.

In mutation profiles, and related methods and systems and labeled genomic components of the present disclosure, repeat regions can be tandem repeats, interspersed repeats, nested tandem repeats, mirror repeats, direct repeats, and/or inverted repeats, and additional repeat regions identifiable a skilled person.

In particular, mutation profiles of the present disclosure can comprise a set of genome values of any one of the above repeat region detected in at least a portion of the genome in of the individual. In particular, in a mutation profile of the present disclosure each genome value of the set is numerically characterized by a value indicative of a first number being representative of a copy number (d) of the repeat region <number of times repeat region is repeated>, and a second number being representative of an error number (m) of the repeat region,

In some embodiments, the repeat regions providing the set of genome value of a mutation profile herein described, comprise tandem repeats. Tandem Repeats are common in both prokaryote and eukaryote genomes. They are present in both coding and non-coding regions and are believed to be the cause of several genetic disorders. The effects of tandem repeats on several biological processes are understood by these disorders. They can result in generation of toxic or malfunctioning proteins, chromosome fragility, expansion diseases, silencing of genes, modulation of transcription and translation [8] and rapid morphological changes [9].

A process that leads to tandem repeats, e.g. through slipped-strand mispairing [2, 10], is called tandem duplication, which allows substrings to be duplicated next to their original position. For example, from the sequence AGTCGTCGCT, a tandem duplication of length 2 can give AGTCGTCGCGCT, which, if followed by a duplication of length 3 may give AGTCGTCGTCGCGCT.

Tandem repeat regions cover about 3% of the human genome. These regions have evolved by a sequence of tandem duplications due to replication slippage events [11, 12] and point mutations (single changes like substitutions, insertions and deletions in the DNA, e.g. ACTG→ACAG). Alone, neither of these metrics provide insight into the genome's rate of change. When viewed together, however, one can learn the relative rates of these mutational events. For example, while point mutations are impossible to detect without reference to an initial genome, their occurrence in repeated regions is indicated by a change in the repeated sequence. Moreover, because the point mutation error is propagated in further repeats, we know exactly when the point mutation occurred relative to the tandem duplications, giving insights into the evolution history of the tandem repeat region. In a sense, tandem repeats are a nature-given repetition error correcting code where point mutation errors in copies store information about the history of the evolution of the region. Furthermore, the duplication rate in tandem repeat regions is very high due to replication slippage events [10], which allows point mutation errors to accumulate, strengthening the evolutionary signal. Hence, tandem repeat regions belong to those markers where we can detect and measure mutation activity.

Tandem repeat regions in the genome can be traced back in time algorithmically to make inference about the effect of the hereditary, environmental and stochastic factors on the mutation rate of the genome. By inferring the evolutionary history of the tandem repeat regions, one can make predictions about the risk of incurring a mutation-based disease, specifically cancer; and more precisely by mutation profiles that are computed without any comparative analysis, but instead are achieved by analyzing the short tandem repeat regions in a single healthy genome and capturing information about the individual's evolution channel. Using gradient boosting on data from more than 5,000 TCGA (The Cancer Genome Atlas) cancer patients, these mutation profiles can, for example, accurately distinguish between patients with various types of cancer.

Even if in the present disclosure, the examples are mainly focused on tandem repeat regions other repeats can be used to provide a mutation profile in the sense of the disclosure as will be understood by a skilled person. In particular, a skilled person would be aware that there is more than 45% of the genome that is covered by interspersed repeats. The information about the duplication history, (m and d values) for these regions can be similarly added to the mutation profile.

An example of both interspersed and tandem duplication of the substring TC of duplication length 2 is

Interspersed:
AGTCGAT → AGTCGATCT;
Tandem:
AGTCGAT → AGTCTCGAT.

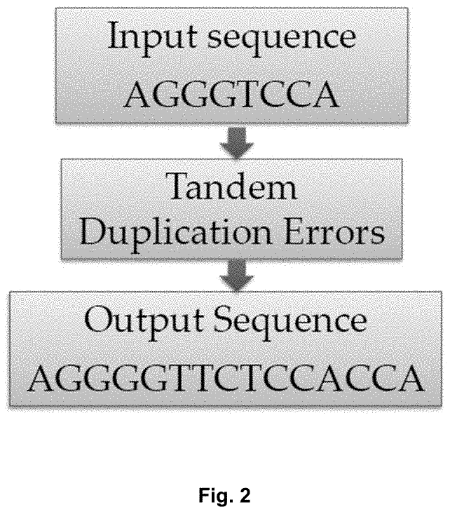

FIG. 2 also shows an example of tandem duplication errors.

In embodiments herein described a method for building a mutation profile for a person can be accomplished by finding at least one repeat region in the DNA and evaluating a consensus pattern for each of the at least one repeat region.

In particular, in methods herein described, finding at least one repeat region in the DNA can be performed by evaluating the DNA sequence and determining if there are any patterns that suggest that some chain of nucleotides in the DNA have at some point in the DNA's history have undergone an event that repeated one or more of the nucleotides in the DNA. Exemplary methods to extract the repeat regions include using software designed to find repeats, such as Benson Tandem Repeat Finder, HipSTR, and GangSTR (for tandem repeat regions) and/or DFAM and RepeatMasker (for interspersed repeat regions). The process of extracting a repeat region identifies the regions the repeat occupies, as well as the initial “seed” sequence that was repeated. Additional techniques that can be used to find repeat regions in genome of the individual are identifiable by a skilled person.

In methods of the present disclosure, evaluating a consensus pattern refers to determining what the original sequence of nucleotides were for a repeat region at some previous point in its history, taken to be considered the “beginning” of the history in question (although not the full history of the entire genome—just a point before the determined duplication and mutation events occurred). A consensus pattern is also known herein as the “seed” for the repeat region in question. For example, a repeat region of “AGGCAGTC” might have a consensus pattern of “AGGC” or “AGTC”, depending on whether the subsequent mutation event (after duplication) was G->T or T->G. Without further information, it might not be determinable which is the actual seed sequence.

Once a consensus pattern has been evaluated, determining a plurality of mutation histories to the at least one repeat region from its corresponding consensus pattern for each of the at least one repeat region can be performed.

The history of repeat regions for a genome refers to the evolutionary list of mutation and duplication events that led some initial seed gene sequence to evolve to its current form, where the current form shows indications that at least one duplication event had occurred during the evolution. A repeat region is a location on a genome where it has been determined that at least one gene duplication event occurred during its history. The evolutionary channel captures the dynamics of the accumulation of point mutations and duplications that occurred from a consensus pattern to a (from the perspective of the consensus pattern) future sequence and history estimation can be considered the same events, but from the perspective of the current sequence looking back and attempting to determine (estimate) the mutation and duplication events that occurred since the consensus pattern.

In some embodiments, finding at least one repeat region in a genomic sequence from the individual; and evaluating a consensus pattern for each of the at least one repeat region, and determining a history of a repeat region numerically can be done by a two-step process. First, apply a repeat finder algorithm, such as the Benson tandem repeat finder [13], to find and extract the repeat regions. Then, use a duplication history estimation algorithm to obtain a pair of numbers—the duplication distance (or “copy number”) d and the mutation distance (or “error number”) m, referred to jointly as the mutation index (m, d) of the region. Instead of using only m and d, one can also accommodate the information about the consensus pattern length and the consensus pattern itself in the mutation history. One can also incorporate the information about the steps in the history where a point mutation occurred, its type (substitution, insertion, or deletion), or other quantifiable information about the mutation history.

An exemplary embodiment is herein described wherein exemplary tandem duplications are detected using Benson's Algorithm.

A tandem duplication is a process which occurs naturally during somatic cell division, in which a short (normally 1 to 6 nucleotides) segment is replicated. For example, the following shows two tandem duplications of length 2, where the duplicated part is indicated in bold. The italicized segment is the so-called microsatellite or repeat region.

CACGTCT ⇒ CAC [symbol] GTCT ⇒ CACGT [symbol] GTCT.

The pattern of a region is the short strand which repeats itself. The copy number d of a repeat region indicates the number of times that the pattern is repeated. For the example given above, the pattern of the italicized repeat region in the right-hand side is GT, and its copy number is 2 (two duplication events).

There are approximately 700,000 microsatellite regions in the human genome, with respective copy numbers of up to 1000. However, they are usually accompanied by various types of errors: substitutions (replacement of one nucleotide by another), deletions (omission of a nucleotide), and insertions (addition of a nucleotide). The total number of substitutions, deletions, and insertions in a repeat region is called the error number m. For example, the following shows the contamination of the previous example microsatellite by 1 substitution, 1 deletion, and 1 insertion:

| | |
|---|---|
| CACGTGTGTCT | ⇒ CACGTG [symbol] GTCT (substitution T→G-in bold) |
| | ⇒ CACGTGGGxCT (deletion T-location indicated with an x) |
| | ⇒ CACGTGGG [symbol] CT (insertion A-in bold) |

Clearly, the copy number here is 2 (from above) and its error number is 3 (three point mutation events), and hence its mutation index is (m, d)=(2, 3). Note that the history of duplication events and mutation events can be interspersed—for example, duplication-mutation-mutation-duplication-mutation, which would result in a different repeat region pattern but the same mutation index. In the first step of Part A, the Benson [13] and Tang et al. [14] algorithms are performed over the entire genome of each patient in the dataset. For each individual genome, we obtain the respective (m, d) values of all repeat regions that were observed, which together constitute the mutation profile of the individual.

Given the aligned vectors, the patterns are omitted, and every ‘-’ is replaced by (0, 0). This results in vectors of identical length. This allows one to come up with a classifier (see the machine learning examples herein) that is capable of estimating a person's inclination to have any type of cancer or a disease in general (as explained herein).

A genome, or a part of a genome, can then be characterized by being further labeled by a list of all the mutation indexes. In one embodiment, the mutation indexes can be labeled by their seeds. For example, consider the following two patients, in which the repeat regions are italicized.

### Patient 1:

AAAAAAACGATCGAGTTCAGTATTGCCGCGAGCG =⇒ (A: (0, 7),
CG: (1, 4))

### Patient 2:

AAAAAAAACGACGTACGTACGTATTGCCGCGCG =⇒ (A: (0, 8),
CGTA: (0, 3), CG: (0, 3))

Each sequence has a corresponding tensor that provides all the mutation indexes identified in that sequence. This is known as a “mutation profile” herein. For comparison purposes, the tensors can be aligned to have equal dimensions. This can be done by a dynamic programming alignment algorithm. In this algorithm, a similarity score is computed recursively for each possible alignment, and the alignment which leads to the best possible score is chosen. Each possible alignment is defined as the sum of normalized edit-distances (that is, the minimal number of insertions, deletions, and substitutions that are required to transform one pattern to the other, divided by the average length of the sequences) between the patterns of all respective pairs. Further, the distance between any pattern and a “missing pattern”, denoted by ‘-’ below, is defined as 0.4. Namely, two patterns whose respective normalized edit distance is less than 0.4 were considered to be equal for the sake of the alignment. For example, the vectors above can be aligned in the following way:

### (A: (0, 7), CG: (1, 4))==⇒(A: (0, 7), -, CG: (1, 4))

### (A: (0, 8), CGTA: (0, 3), CG: (1, 3))==⇒(A: (0, 8), CGTA: (0, 3), CG: (1, 3))

The score for the above alignment is dₑ(A, A)+dₑ(-, CGTA)+dₑ(CG+CG)=0+0.4+0=0.4, where dₑ is the edit distance. For comparison, the alternative alignment:

### (A: (0, 7), CG: (1, 4)==⇒(A: (0, 7), CG: (1, 4), -)

(A: (0, 8), CGTA: (0, 3), CG: (1, 3))==⇒(A: (0, 8), CGTA: (0, 3), CG: (1, 3)) has score dₑ(A, A)+dₑ(CG, CGTA)+dₑ(-, CG)=0+2/3+0.4 1.06, and hence is preferred over the previous alignment as reflected by the higher score.

One can infer the mutation history of individuals from a DNA snapshot of their repeat regions; a task that one might describe as an equivalent to inferring a short video by observing the last image in it. However, any repeat region might have multiple possible mutation histories, each of which produces potentially different mutation number m and duplication number d. Hence, it is crucial to devise a rigorous method of coming up with only one pair (m, d) from any given region, that best describes the entire space of possible histories.

One embodiment of the algorithm ranks the possible histories according to the energy that is required to obtain them, a quantity that corresponds to the probability of occurrence. Then, the history with the lowest amount of energy is chosen, and the respective (m,d) are computed exclusively from it. In a further embodiment, an alternative method that has a greater potential for encapsulating the space of possible histories and suggests a smaller risk for propagation of errors due to inaccuracies can be achieved with an extended algorithm.

Extended Algorithm: Instead of choosing the history with the smallest energy, match each possible history i to a number nᵢ which reflects the energy amount in a decreasing order. For example:

The lowest energy history gets 1, the second lowest gets 0.9, and so on; or every possible history gets its relative reciprocal energy, i.e., history i gets

$$\frac{1 / {e}_{i}}{{\sum }_{j} 1 / {e}_{j}} ,$$

where eⱼ is the energy of history j.

Then, the respective (mᵢ, dᵢ) are computed for every history i, and the final result is

$$( m , d ) = \frac{{\sum }_{i} {n}_{i} ( {m}_{i} , {d}_{i} )}{{\sum }_{i} {n}_{i}} .$$

This alternative method reflects all possible histories, where the more probable ones are given higher weights than less probable ones. The least energetic history is still the most influential one, but other histories are not discarded, and can still affect the final outcome.

In methods of the present disclosure, determining a plurality of mutation histories can be performed by taking the consensus pattern and the resulting current sequence that evolved from the consensus pattern, and considering a number of different ways the consensus pattern could have resulted in the current sequence, in terms of duplication and mutation events. In one embodiment, all possible histories are considered. In another embodiment, only a subset of histories can be considered.

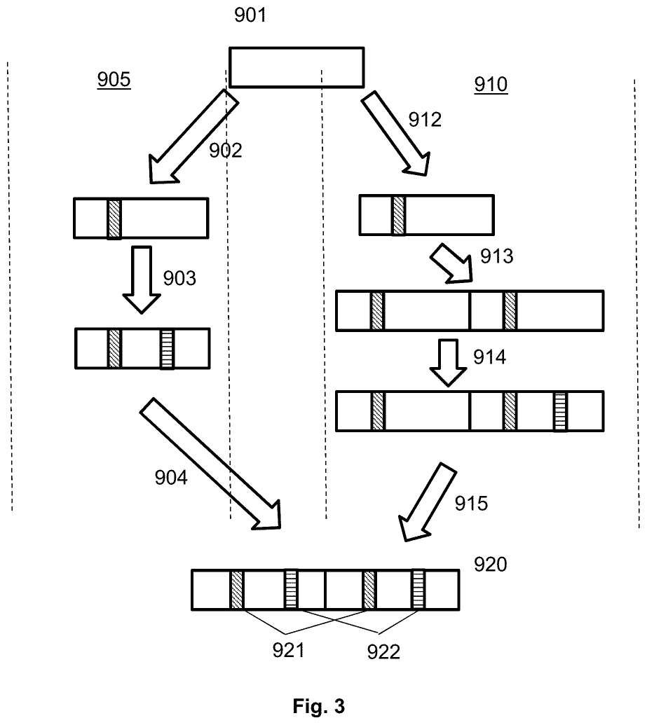

FIG. 3 shows an example of two possible histories of an evolutionary channel. Given a present sequence (920) that has apparent repeat regions with two point mutations in each repeated region, each pair matching location, type, and nucleotide in each region (for example, pair of matching substitution G->C (921) and a pair of matching insertion T (922) mutations). The “seed” sequence would be the non-duplicated, non-mutated sequence (901), but there are a number of different ways that the seed sequence (901) could have reached the current sequence (920). Ideally, sequencing snapshots would have been taken during the patient's lifetime, but that is unlikely to be available. Therefore, the history of the sequence has to be estimated. One possible history (905) includes a substitution mutation (902) followed by an insertion mutation (903), then a final duplication of the region (904). Another history (910) is a substitution (912), followed by a duplication (913), then an insertion (914), followed by another insertion (915). These, of course, are not the only two possible histories. Comparing the two, it is evident that the first possibility (905) is more likely than the other (910), because the second possibility requires two separate insertions (914, 915) matching location, type, and nucleotide in each repeated region. Another way of looking at the comparison is that the first history (905) requires less energy than the second history (910). When considering the evolutionary channel, one can consider the most likely history (the one with the least energy requirement). Alternatively, one could consider a superposition of all the histories, but that would require more bandwidth to store that information. In one embodiment of the superposition, the histories can be weighted based on their likelihood/energy.

The History Estimation approach is not restricted to tandem repeats and can also be used for other structural variations in the DNA, for example interspersed repeats, and additional repeats herein described. Further ideas from phylogeny estimation can also be used for history inference in those scenarios [15].

One metric for the histories is the history length. This is the count of the steps it takes to go from seed to target sequence for a particular history. As seen below, it can be useful to separate the history length into “duplication count” (the count of the duplication steps) and “mutation count” (the count of the point mutations, i.e. total of substitutions, insertions, and deletions). In a further embodiment, the mutation count can be separated to “substitution count”, “insertion count”, and “deletion count”, to give counts of each type of mutation separately. Further, the information encapsulating the location of point mutations and number of point mutations per base can also be included.

In some embodiments, identifying a history ranking and representation of one or more seed can comprise performing a history estimation algorithm.

Given a consensus pattern P and a repeat R, find the most likely path of generating R from P. The repeat R can be represented as P₁P₂P₃ . . . P_{N} where each Pᵢ is at a edit distance of d(P, Pᵢ) from P. “Edit distance” as used herein means how dissimilar two sequences are by a count of how many operations are needed to transform one sequence to another. The edit distance can be calculated using the Smith-Waterman algorithm [16]. In Smith-Waterman algorithm, it is assumed that d(x,y)=w_{xy}, where xε{A,C,G,T,-} and yε{A,C,G,T, -} and x≠y and wₓₓ=0. Each w_{xy} can take different values depending on the biological constraints. In this example it is assumed that wₓₓ=1 and the consensus pattern P is obtained from Benson TRF which uses Method 1 [17]. For the history estimation algorithm,

Input: P, P₁P₂P₃ . . . P_{N} and w_{xy}
Objective: Minimize the cost of generating P₁P₂P₃ . . . P_{N} from P—Cost(P, P₁P₂P₃ . . . P_{N})

Assumption: In each step only one block can be tandemly duplicated with point mutations. i.e. tandem duplication of Pᵢ [symbol] PᵢPⱼ or Pᵢ [symbol] PⱼPᵢ is allowed but tandem duplication of PᵢPⱼ [symbol] PᵢPⱼPₖPₗ or PᵢPⱼ [symbol] PₖPₗPᵢPⱼ is not allowed in one step.

Main idea: Divide the repeat into 2 partitions—P₁P₂P₃ . . . Pₖ and Pₖ₊₁Pₖ₊₂ . . . P_{N}

Cost(P,P₁P₂P₃. . . P_{N})=min_{u,v,k:1≤u≤k,k+1≤v≤N} Cost(Pᵤ,P₁P₂P₃. . . Pₖ)+Cost(Pᵥ,Pₖ₊₁Pₖ₊₂. . . P_{N})+Cost(Pᵤ,Pᵥ)
With Cost(Pᵢ, PᵢPⱼ)=d(Pᵢ,Pⱼ) and Cost(Pᵢ, PⱼPᵢ)=d(Pᵢ,Pⱼ)

Dynamic programming can be used in this example to find the minimum cost. This algorithm is given in Tang et al. [18].

The assumption of a single block duplication in each step can be replaced by a multiblock duplication, however the algorithm for that is based on heuristics and is not optimal. One such algorithm with the name of WINDOW algorithm is presented in Tang et al. [14].

Also the assumption that Pᵢ [symbol] PᵢPⱼ can be replaced by Pᵢ [symbol] Pᵢ′Pⱼ where Pᵢ′ is different from Pᵢ An optimal version of an algorithm for this setup is also presented in Tang et al. [14].

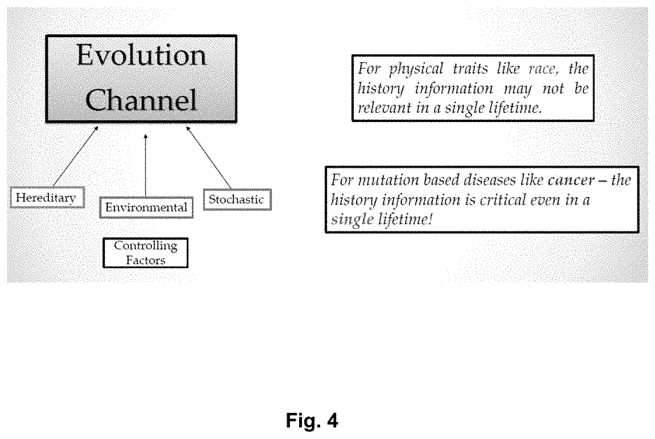

FIG. 4 shows the controlling factors for the evolutionary channel, namely hereditary, environmental, and stochastic (random) factors. Note that for physical traits like hair color, the history information may not be relevant in a single lifetime, whereas for mutation-based diseases, like cancer, the history information can be critical even within a single lifetime.

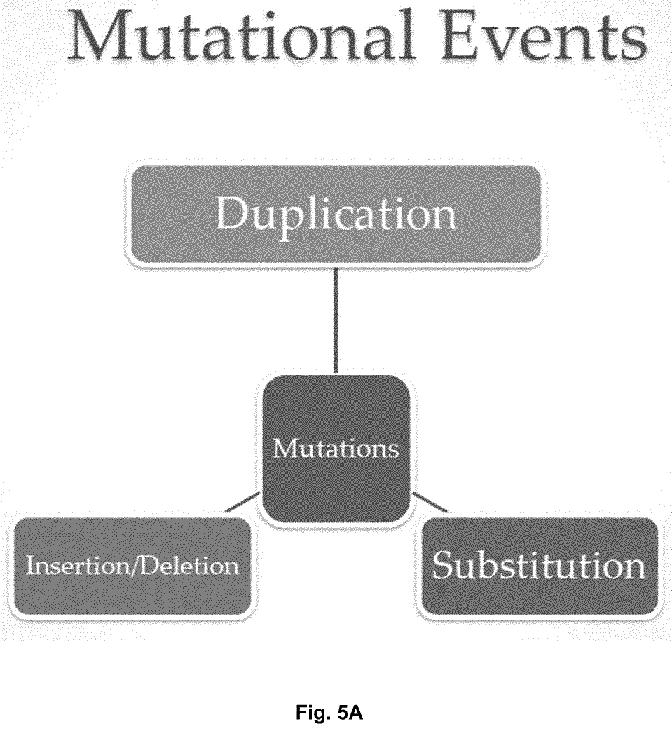

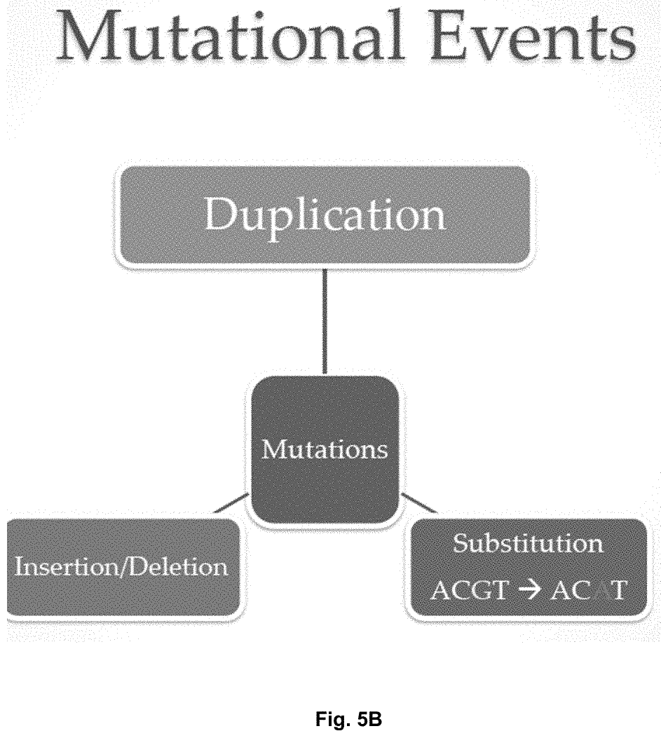

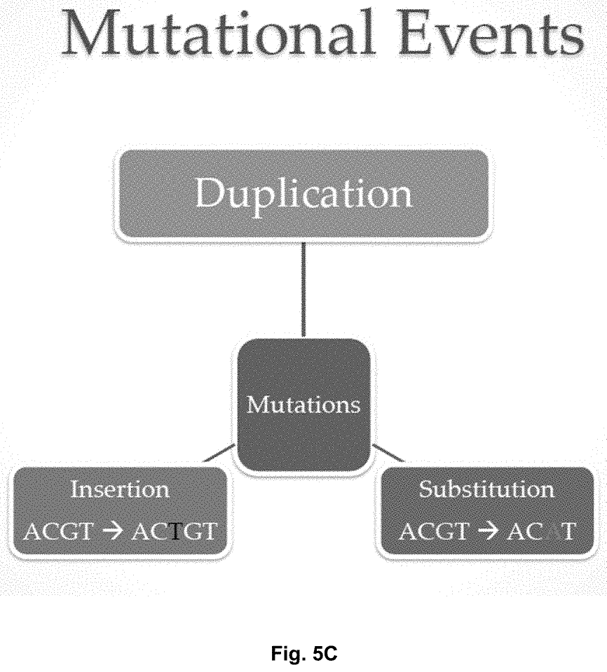

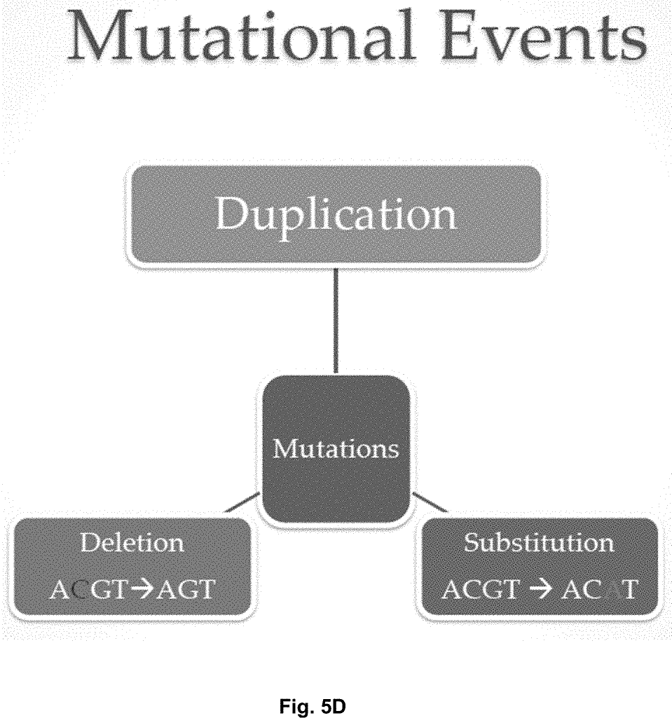

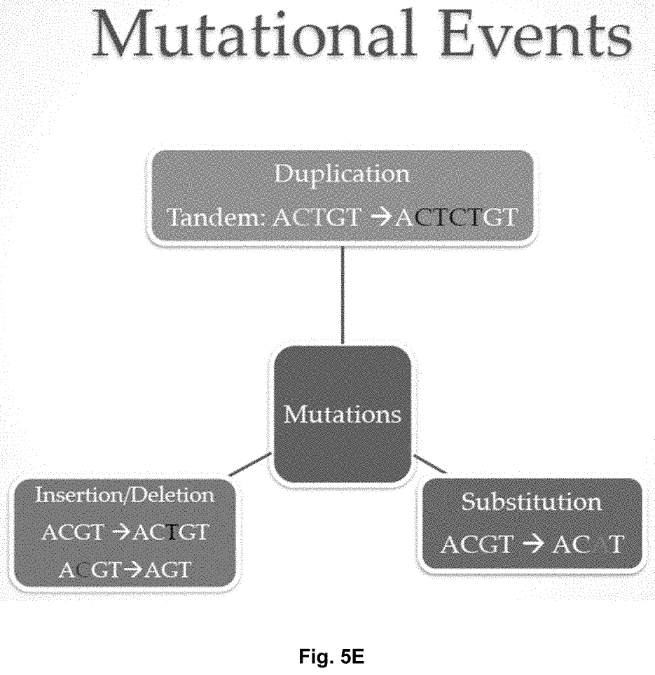

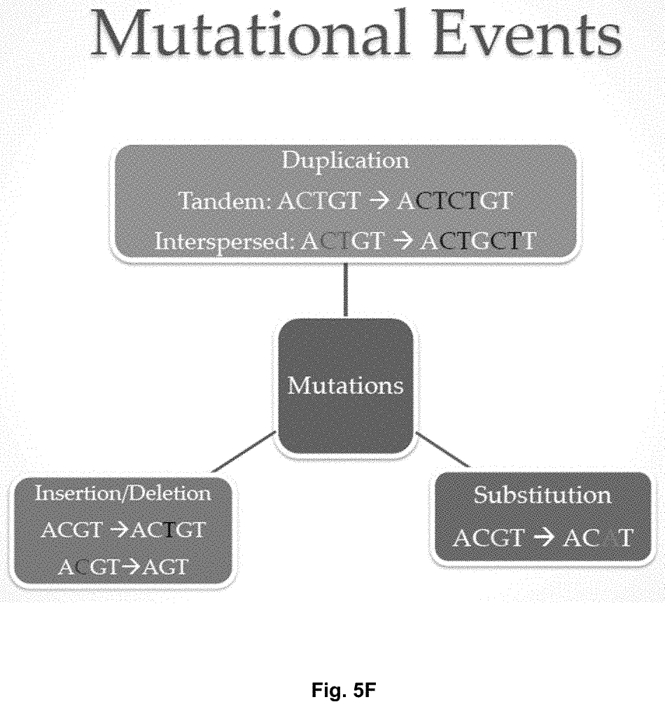

FIGS. 5A-5F show various types of mutational events, specifically duplication, insertion, deletion, and substation.

In particular, in vectors, methods and systems herein described, a mutation accumulation is governed by an underlying evolution channel, wherein the term channel is motivated by the introduction of channels in the theory of communication by Shannon [18] in 1948). The evolution channel is controlled by hereditary, environmental, and stochastic factors.

Once the plurality of mutation histories has been determined, building a mutation profile based on at least one mutation history of the plurality of mutation histories can be performed, the mutation profile comprising a mutation index for each consensus pattern. In embodiments, herein described, following performing history ranking and representation, methods to build a mutation profile comprises labeling the repeat region to characterize history of the repeat region numerically.

The mutation profile can be then constructed by associating a mutation index corresponding to the most probable mutation history for each of the at least one repeat regions.

In some embodiments in a method for building a mutation profile for an individual the finding can be preceded by sequencing DNA from the individual.

In some embodiments, sequencing DNA from a person can be performed by taking a tissue sample from a person and then subjecting the tissue to a DNA sequencing technique, such as Maxam-Gilbert, chain-termination, shotgun sequencing, bridge PCR, ion semiconductor, pyrosequencing, combinatorial probe anchor synthesis, ligation, nanopore, or any other method that would result in a sequence of the portion of DNA of interest, or of the entire genome.

In preferred embodiments, to reduce bias, the sequencing technique, amplification technique, and sample tissue type are uniform for training, testing, and diagnosing. In those embodiments, different sample tissue types (e.g. blood, liver, lung, etc.), can either be used for separate classifiers (e.g. blood sample brain vs. liver classifier and liver sample brain vs. liver classifier), or a tissue type variable can be included as an element in the mutation index. If separate classifiers are used, a final determination of risk propensity can be determined by either combining the propensities of the different tissue sample types (e.g. averaging or otherwise statistically combining) or by considering how many classifiers produce a high risk (higher than the other condition types) and if a majority show high risk, then diagnosing the condition as being the prevailing risk.

For a classifier built/using mixed tissue sample locations (e.g. both blood and skin samples used in the data for one classifier), one should first determine if there is an inherent bias between the two tissue types. One way to determine this is to see if the accuracy on the diagonal (e.g. brain cancer vs. brain cancer, pancreatic cancer vs. pancreatic cancer, etc.) is close to 0.5 (50%).

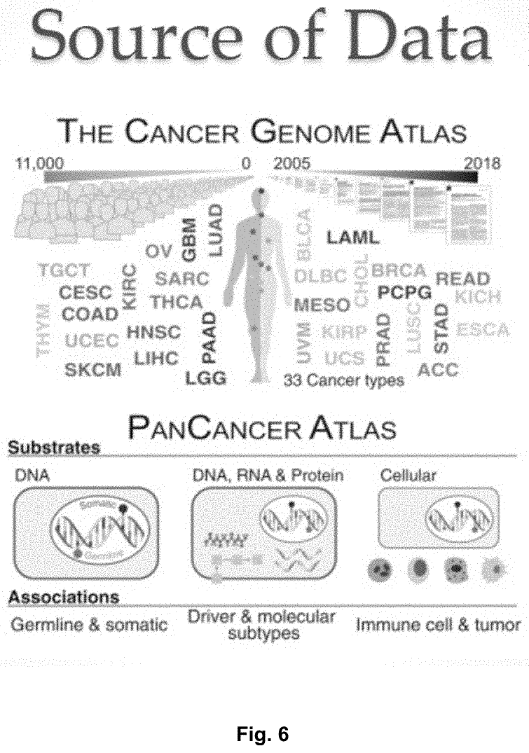

FIG. 6 shows examples of sources of sequencing data. Examples include high quality (30-40× coverage) Whole Exome Sequencing Data (WES) can be obtained for about 11000 cancer patients covering 33 different cancer types from The Cancer Genome Atlas (TCGA). The data comprises DNA derived from tumor cell, normal matched to tumor and the blood cell for each patient. Other sources of Cancer data are International Cancer Genome Consortium (ICGC). ICGC consists of Whole Genome Sequencing (WGS) data for about 2500 patients.

Both in embodiments comprising sequencing and in embodiments not comprising the sequencing, a method for building a mutation profile for an individual results in a mutation profile of the individual indicative of development and diversification of the genome of the individual in time.

The term “profile” as used herein indicates a set of data that portrays significant features of a referenced item. As a consequence, the term mutation profile of an individual in the sense of the disclosure indicates a set of data indicating significant features of the mutations characterizing at least a portion of the genome of the individual. It can also be thought of as a signature that characterizes individual's genome. A mutation profile of an individual is therefore indicative of development and diversification of the genome of the individual in time. and of the effect of such development and diversification on the individual, such as the propensity of the individual to develop a condition.

A mutation profile according to the present disclosure is a profile that comprises a set of genome values representing history of repeat regions of at least a portion of the genome in of the individual.

A set of genome values is a vector, matrix, tensor, or the like containing values associated with a genome, for example associated with regions in the genome. Each value (data object) having two or more scalar quantities associated with some aspect of a genome. In theory, each value can have only one quantity associated with it, but two or more is preferable. For example, a vector of mutation (m) and duplication (d) events in a genome's history can be represented as a list of m and d values for various portions of the genome, which can be visualized as ((m₁, d₁), (m₂, d₂), (m₃, d₃), . . . (mₙ, dₙ)) for n regions of the genome, which would be a two dimensional vector mutation profile. Likewise, the same values could also be represented by a 2×n matrix, or other forms as shown herein.

The data object's elements can include information about the evolutionary history of the repeat region in question, such as the number of duplication events, the number of mutation events, the length of the repeat region, the location of the repeat region in the genome, the positions of the point mutations, the graphical structure of a history estimation graph, the weighted sum of different history paths, and/or any value describing an aspect of estimated histories of the repeat region. The elements can be unweighted values, weighted values, ratios of two different values (for example, the ratio of mutation events to duplication events), or average values over multiple histories. The elements can be based on multiple histories or a representative history, such as a history estimated to have the lowest energy cost for producing the repeat region. The data object for the i-th repeat region can be referred to as mutation index Rᵢ. An example data object containing information about the number of mutation events m and the number of duplication events d for a representative history of a region i can be expressed as Rᵢ=(mᵢ, dᵢ).

Other examples of data objects related to evolution histories:
Rᵢ=(mᵢ, dᵢ) where mᵢ is the number of point mutation events and dᵢ is the number of duplication events in the history with the shortest path (i.e. m+d is minimized).
Rᵢ=(mᵢ, dᵢ) where mᵢ is the average number of point mutations and dᵢ is the average number of duplications, the average is taken over all possible mutation histories, with probabilities proportional to the likelihood of each history.
Rᵢ=(mᵢ, dᵢ, lᵢ) where is the length of the seed or the repeated pattern.
Rᵢ=(mᵢ, dᵢ, aᵢ, cᵢ, gᵢ, tᵢ) where aᵢ, cᵢ, gᵢ, tᵢ are the number of A, C, G and T nucleotides, respectively, in the seed for repeat region i.
Rᵢ=(mᵢ, dᵢ, aᵢ,cᵢ, gᵢ, tᵢ, MLᵢ) where MLᵢ represents the methylation level of repeat region i in the genome.
Rᵢ=(mᵢ, dᵢ, Vᵢ) where Vᵢ stores the locations at which point mutations occur in the history.

Additionally, the values can be weighed by other factors. For example, the copy number d can be multiplied by a weight based on what type of duplication is being counted (tandem, interspersed, etc.). Alternatively, the weighting factor can just be an added element to the mutation index (e.g. (mᵢ, dᵢ, tyᵢ), where ty is a value associated with duplication type). The weights/added elements can also be based on environmental and/or behavioral information (e.g. age, smoking habits, diet, geographic location, etc.).

In certain embodiments herein described each multidimensional genome value being numerically characterized by a value indicative of a first number being representative of a copy number (d) of the repeat region, and a second number being representative of an error number (m) of the repeat region.

A copy number of a repeat region is a count of the number of times pattern duplication is believed to have occurred for a given repeat region during its history. The duplication events do not need to duplicate the exact same set of nucleotides to be counted.

An error number of a repeat region is a count of point mutation events believed to have occurred in the repeated regions during its history. Point mutations include deletions, insertions, and substitutions of individual nucleotides.

A mutation profile in the sense of the disclosure is indicative of development and diversification of the genome of the individual in time. In particular, the mutation profile in the sense of the disclosure conveys the evolution history of the DNA of the individual, considering the occurrence of mutations and duplications over time as an evolutionary channel reflecting an estimation of the history of the genome.

In embodiments herein described a mutation profile can be used to provide a labeled human genome component, comprising at least a portion of a genome of an individual in combination with the mutation profile. At least a portion of a genome refers to any number of nucleotides of a genome, up to and including the full genome.

In embodiments herein described a mutation profile can be used in a method of predicting a risk of occurrence of a target condition in an individual, the target condition associated with genomic factors can be realized. The method can include: detecting, in a healthy cell of the individual, a mutation profile, the detected mutation profile indicative of development and diversification of the genome of the individual in time; and comparing the detected mutation profile with a reference mutation profile associated with the condition to provide a condition risk propensity for the individual.

These mutation profiles, taken from a population of profiled individuals, can be used with machine learning to build a classifier to classify any shared phenotype of that population. In exemplary embodiments herein described cancer is as an example condition, but any other condition associated to genomic factors can be used as will be understood by a skilled person upon reading of the present disclosure. The difference would be in how far back the histories go—a condition like such as heart disease, high blood pressure, stroke, and diabetes. would have histories that could go back generations. However, many conditions, like some types of cancer, can usually be traced back within the lifetime of the individual in question.

Cancer is currently the leading cause of death worldwide [19]. Yet, cancer is caused by an intricate mixture of complex factors, and their inter-relations are not well understood. Traditionally, cancer is attributed to either the Environmental (E) or the Heredity (H) factors [3], but the aforementioned breakthrough in etiology of cancer [3] suggests that random mutations (R), might have a significant impact in various lethal instances of the disease (see FIG. 1). It was also demonstrated in [3] that certain cancer types (such as lung or skin) correlate well with E mutations, whereas others (such as prostate, brain, or pancreas) correlate well with R mutations.

Notwithstanding its generality, the applicability of this approach on the special case of cancer prediction was incentivized by a few independent factors. First, a large body of recent research (see above) indicates a high correlation to random mutations, and hence some cancer types might be able to serve as a test bed for these techniques.

Second, data-driven approaches are of little to no merit when data is scarce. This is certainly not the case when it comes to cancer, as a plethora of well-labelled high-quality whole genome datasets are readily available in the The Cancer Genome Atlas (TCGA) [20]. TCGA database consists of high-quality individual genome data for 33 different cancer types. Full genome extracted from normal or healthy cell and tumor cell for each cancer patient is available on this database.

By combining approaches from coding theory, combinatorial algorithms, and machine learning, an algorithm for classifying an individual's personal mutation mechanism is devised, along with its correlation with various diseases. In this algorithm, the DNA of a healthy cell of an individual is analyzed and its mutation profile is estimated. Then, by applying a pre-trained classifier on this mutation profile, the individual's inclination to develop several diseases is determined.

For example, a method for determining a condition risk propensity for a target condition in an individual can be performed. The method can include: determining a first set of mutation profiles for a population of individuals with the target condition, each mutation profile of the first set of mutation profiles being a mutation profile for each corresponding individual of the population of individuals with the target condition; determining a second set of mutation profiles for a population of individuals not having the target condition, each mutation profile of the second set of mutation profiles being a mutation profile for each corresponding individual of the population of individuals not having the target condition.

A “target condition” is a condition (e.g. disease) of interest. It is possible that multiple target conditions are to be tested at the same time. For example, all pulmonary system related cancers might be of interest, such as lung cancer, squamous cell cancer, and heart cancer. Or, maybe all cancers are of interest for a general screening.

The term “population” indicates any number of individuals typically of a same tassonomical group and in particular a same species. In preferred embodiments, a population indicates humans. In some embodiments, individuals forming the population can be selected based on presence of additional common genetic traits and other common features such as race, ethnicity and/or geographic location.

The method then includes training a classifier using the first set of mutation profiles and the second set of mutation profiles; and running the classifier on a mutation profile of the individual such that a risk propensity for the target condition is generated.

“Machine learning” as used herein refers to any method of data analysis that automates analytical model building. Types of machine learning includes neural networks, vector machines, Bayesian networks, genomic/evolutionary algorithms, decision trees, and other known systems.

A “classifier” is an algorithm that implements classification in machine learning. “Classification” refers to identifying which set of categories (e.g. conditions) a sample (e.g. individual) belongs to or how far the sample is from a category. Classifications can be binary (between two categories) or multi-class (between more than two categories at the same time).

Training a classifier consists of using a set of data establish a machine learning model that can accurately classify a new data point.

Running a classifier consists of entering a new data point in the machine learning model and allowing the trained machine learning algorithm to determine the classification of that new data point.

Generating a risk propensity means the creation of a data structure that represents the risk propensity, either for further computing or for display to a user.

In some embodiments, the method can comprise determining a plurality of sets of mutation profiles for a plurality of populations, each of the plurality of populations having a corresponding target condition unique to that population, each mutation profile of the plurality of sets of mutation profiles being a mutation profile for an individual of the plurality of population; training a classifier using the plurality of sets of mutation profiles, classifying by condition; and running the classifier on a mutation profile of the individual such that a risk propensity is generated for the plurality of target conditions. This is an example of using non-binary classifiers (multi-classifiers) trained on multiple populations with different target conditions, which can be used to predict which conditions an individual is most likely to develop over all the conditions.

An algorithm for the methods to predict risk profile of an individual based on mutation profile of the individual herein described allows to achieve important results over existing methods. First, it provides a risk profile based on a rigorous mathematical approach to label a repeat region numerically by a pair of numbers that indicate how noisy its creation process was. Second, it sheds light on the way that these labels are correlated with any particular disease. Third, it constitutes a first-of-its kind predictor which relies on cumulative DNA statistics, rather than the presence or absence of specific genes in specific loci. This suggests an inclusive approach that may extend well beyond any particular application, and any disease which correlates well with high stem cell division can be analyzed similarly [21], [22], [23].

In methods to predict a risk profile herein described determining a mutation profile for an individual or a population of individual can be performed by a two-step process. First, apply a variant of the well-known Benson tandem-repeat finder algorithm [13] to extract the repeat regions. Then, use the duplication history estimation algorithm by [14] to obtain a pair of numbers—the copy number d and the error number m, referred to jointly as the mutation index (m, d) of the region. This process can be applied on a healthy cell genome of every member in a dataset of sick individuals, and each of which is consequently mapped to a vector which contains the (m, d) values for all repeat region in their DNA. These vectors, called mutation profiles, are aligned and given as a training set for a learning algorithm that outputs a prediction model which provides a disease risk propensity whose accuracy is estimated by cross-validation. In the case of cancer, this risk propensity is called a cancer risk propensity.

A “risk propensity” as used herein refers to a list of one or more conditions and their corresponding probability of occurrence.

An example Workflow with Machine Learning is provided herein below.

In the exemplary workflow, the algorithm can be partitioned to Part A (building the classifier) and Part B (using the classifier). Part A can be only performed once, whereas Part B is performed whenever cancer or in general disease prediction is required. In Part A, a dataset of healthy cell DNA from individuals is first processed by the Benson [13] and Tang et al. [14] algorithms to deduce the mutation profiles for all individuals. Then, these vectors are aligned by a dynamic programming algorithm to resolve missing regions issues. Finally, the aligned vectors are fed into a training algorithm that produces a classifier. In Part B, this classifier is applied over any individual's genome, to assess the overall risk to contract any of the diseases in question.

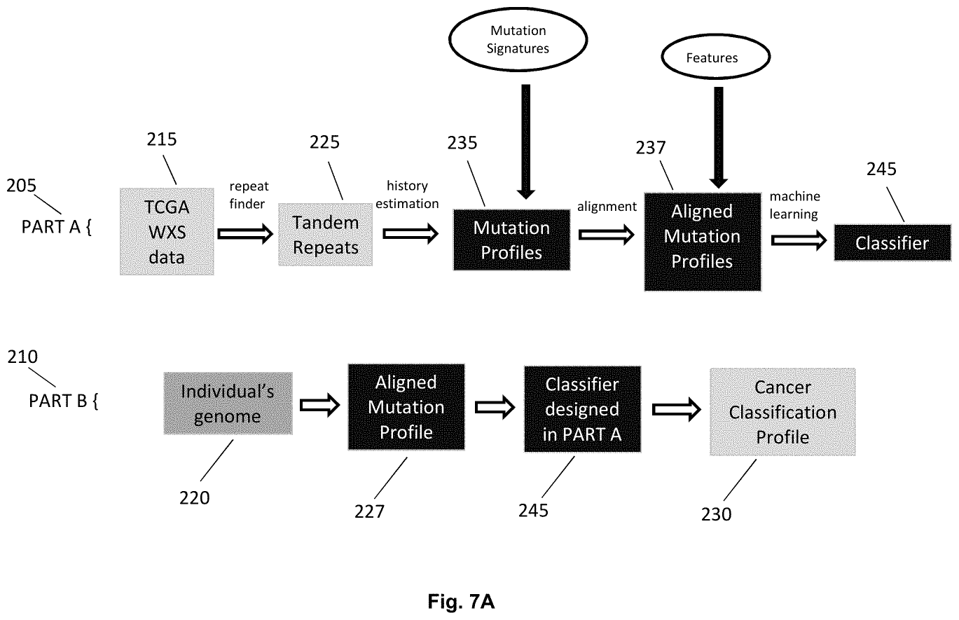

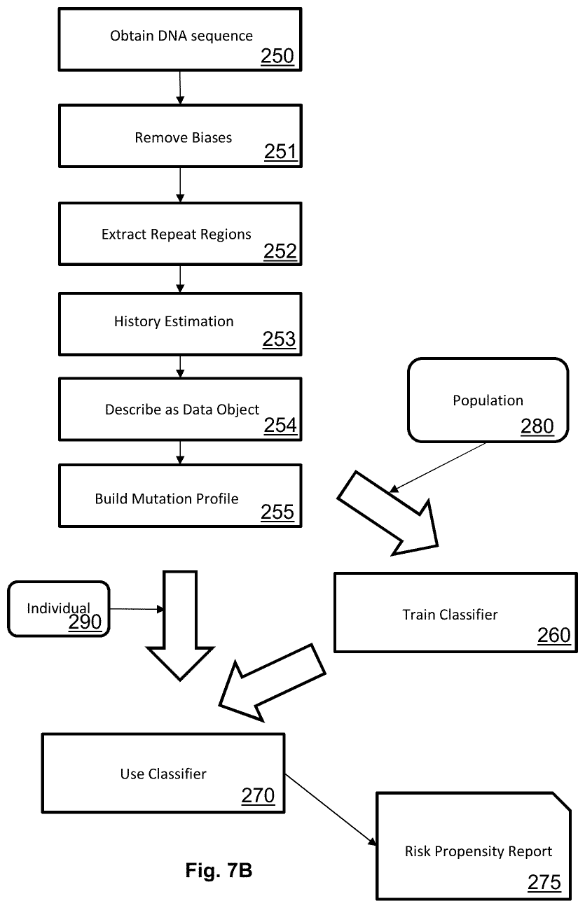

FIG. 7A shows an example workflow for an embodiment of the present disclosure. In Part A (205) a classifier (245) is trained based on aligned mutation profiles (237) by developing mutation profiles (235) from a data set of known cancer patients (215) based on tandem repeat regions found (225) in their DNA. In Part B (210), the resulting classifier (245) is applied over an aligned mutation profile (227) from an individual's genome (220) to assess that individual's inclination of developing cancer in a disease risk propensity (230).

FIG. 7B shows another example workflow for an embodiment of the present disclosure. In one embodiment, this approach can be provided in a number of steps to build a “mutation profile”.

Step 0 is the preparation of the inputs to the system. DNA sequences would be obtained (250) either by sequencing DNA from biological samples or by downloading the sequences from a database (or a combination of the two). To increase the accuracy of the sequences, methods to remove bias and/or purify the sample (251) such as PoN filtering [24] to remove technology and site-specific artifacts, ContEst [25] to assess sample contamination, and the removal of potential DNA oxidation artifacts [26] can be used.

Step 1 is extracting the repeat regions (252) (e.g. tandem repeats, interspersed repeats, nested tandem repeats, mirror repeats, direct repeats, and/or inverted repeats, et al.) from the DNA sequences. Examples of methods to extract the repeat regions include using software designed to find repeats, such as Benson Tandem Repeat Finder, HipSTR, and GangSTR (for tandem repeat regions) and/or DFAM and RepeatMasker (for interspersed repeat regions). The process of extracting a repeat region identifies the regions the repeat occupies, as well as the initial “seed” sequence that was repeated.

Step 2 is estimating the histories of the repeat regions (253). Examples of estimating tandem repeat regions include the methods proposed by Tang et al. [27] and Farnoud et al. [28]. An example of estimating for interspersed repeats includes phylogeny methods [15].

Step 3 is describing the estimated histories as a data object (254). The data object's elements can include information about the evolutionary history of the repeat region in question, such as the number of duplication events, the number of mutation events, the length of the repeat region, the location of the repeat region in the genome, the positions of the point mutations, the graphical structure of a history estimation graph, the weighted sum of different history paths, and/or any value describing an aspect of estimated histories of the repeat region. The elements can be unweighted values, weighted values, ratios of two different values (for example, the ratio of mutation events to duplication events), or average values over multiple histories. The elements can be based on multiple histories or a representative history, such as a history estimated to have the lowest energy cost for producing the repeat region. The data object for the i-th repeat region can be referred to as mutation index Rᵢ. An example data object containing information about the number of mutation events m and the number of duplication events d for a representative history of a region i can be expressed as Rᵢ=(mᵢ, dᵢ).

Step 4 is aggregating the data objects into a mutation profile (255). For n extracted repeat regions where Rᵢ represents a mutation index that stores information about the evolution history of repeat region i, the mutation profile can be represented as profile P={Rᵢ}ᵢ₌₁ⁿ which aggregates evolution history information of all extracted repeat regions from i=1 to n for a given DNA sequence. The profile does not necessarily contain all the possible evolution history information, but for many applications even a limited amount of information can be useful.

One use of a mutation profile is to make comparisons of the mutation profiles of different individuals in a population (280) to train a classification system (260) for different conditions (e.g. brain cancer, prostate cancer, Alzheimer, heart disease, autoimmune disease, etc.). This classification system can be implemented through signal processing, statistical, and machine learning methods to derive a “model” (or “classifier”, or “differentiator”) which associates mutation profiles with a propensity to incur a condition. One statistical method to achieve this model would be to use mutation profiles of these individuals as features and the condition they incur as the target as part of training data to build a machine learning based classifier that associates risk for different conditions with the mutation profile. Because the classification is looking at general evolution history information, and not necessarily direct mutation causal information, the mutation profiles can be built from DNA extracted from healthy tissue. Machine learning algorithms like SVM, logistic regression, Gradient boosting, random forest, neural networks, etc. can be used to build the classifiers. Both pairwise and multi classifiers can be built.

With a built classifier, the model can be used (270) to classify a mutation profile from an individual (290) who has not yet been diagnosed with a condition of the classifier to determine that individual's risk propensity (275) for that condition (or conditions).

In particular for the cancer related case study, healthy-cell genomes from The Cancer Genome Atlas (TCGA) of patients with either lung, squamous cell lung, brain, prostate, pancreas, or stomach cancer are obtained, and their mutation profiles are extracted by applying the first two steps of Part A (Benson's Algorithm and Alignment). Then, a binary classifier can be trained for every pair of types of cancer, generating 15 classifiers overall. The confidence levels in either of those classifiers is used as a measure for the “uniqueness” of the mutation profiles that cause a certain type of cancer and can additionally be seen as a distance measure between different types of cancer.

This approach yields a series of pairwise classification algorithms that, in turn, can be applied over any individual genome to predict if they are more inclined to one than to the other. However, providing an overall measure which indicates the individual's inclination for all types of diseases in question simultaneously can be achieved by applying all pairwise classifiers on a given genome, thereby aggregating their prediction results into a single estimation vector called the disease risk propensity.

In some embodiments, classification can be performed through rank aggregation. In those embodiments, Part A of the algorithm results in a series of binary classifiers. One embodiment of this algorithm is a simple algorithm which combines these binary classifiers. In particular, to obtain a classifier that is as informative as possible regarding all the diseases in question and hence creating a disease risk propensity.

The simple algorithm consists of two parts. Given a genome of a patient, in the first part of the algorithm apply each one of the binary classifiers, which results in a series pairwise ranks, that indicate if the patient is more inclined to develop one disease or the other. Then, in the second part these ranks are aggregated to form a list of the diseases in question, sorted from least to most likely.

Mathematically, these ranks can be seen as inequalities between the different diseases. For example, if a certain classifier aims to distinguish between a person's susceptibility to develop LUNG-CANCER or ALZHEIMER, and its output on the given patient is ALZHEIMER, we say that LUNG-CANCER<ALZHEIMER. Repeating this process over every pair of diseases, a set of inequalities can be obtained, as in the following example.

### Diseases: {LUNG-CANCER, ALZHEIMER, MELANOMA, PROSTATE-CANCER}

### Binary Classification Results:

### PROSTATE-CANCER<MELANOMA

### PROSTATE-CANCER<LUNG-CANCER

### PROSTATE-CANCER<ALZHEIMER

### MELANOMA<LUNG-CANCER

### MELANOMA<ALZHEIMER

### LUNG-CANCER<ALZHEIMER.

In this case, it is readily verified that the above 6 pairwise classifications can be aggregated as:

### PROSTATE-CANCER<MELANOMA<LUNG-CANCER<ALZHEIMER,

which is the output of Part B. Namely, the algorithm in this case determines that the given patient is most likely to develop ALZHEIMER, and least likely to develop PROSTATE-CANCER.

However, due to the imprecise nature of data-driven techniques, it is occasionally the case that the pairwise inequalities are not consistent with any overall ranking, for instance,

### Diseases: {STOMACH-CANCER, LEUKEMIA, BRAIN-CANCER}

Binary classification results:

### STOMACH-CANCER<LEUKEMIA

### LEUKEMIA<BRAIN-CANCER

### BRAIN-CANCER<STOMACH-CANCER,

where it is evident that the induced ordering is circular, and hence no coherent linear ordering is possible. If this happens to be the case, there exists a rich literature (e.g., [29][30][31] and references therein) about finding a linear ordering which minimizes the number of pairwise errors. That is, the confidence levels of the binary classifiers are seen as “penalties”, and a given linear ordering is scored by the sum of confidence levels of the pairs that are incorrectly ordered. For instance, if the confidence levels of the binary classifiers in the above example are 1, 2, and 3, respectively, then the penalty of the ordering

### STOMACH-CANCER<LEUKEMIA<BRAIN-CANCER

is 3, since only the pair (STOMACH-CANCER, BRAIN-CANCER) is incorrectly placed. For comparison, the penalty of the ordering

### LEUKEMIA<BRAIN-CANCER<STOMACH-CANCER

is only 1, and hence it would be preferred over the previous ordering.

Validation can then be performed with various approaches identifiable by a skilled person.

For example, one way to validate the classifier is by cross-validation. An example of cross-validation is 4-fold cross-validation. In 4-fold cross validation, the data for a classification pair (for example, stomach cancer vs. brain cancer) is randomly split between four groups equally: A, B, C, and D. Then four classification-test rounds are performed on the machine learning model: one where A is the test data and B+C+D are used to train the classifier, one where B is the test data and A+C+D are used to train, one where C is the test data and A+B+D are used to train, and one where D is the test data and A+B+C are used to train. This reduces the chance that the accuracy of the model for that pair is not skewed based on the distribution of the data between training and test. This also ensures that the classifier is being tested on data it has not seen in other instances of testing, which helps prevent overfitting. This validation can be extended to any number (k-fold cross-validation), by just dividing the data into a different number of groups (k groups) and running more iterations of classification+test (k iterations).

In embodiments herein described wherein the condition comprises a cancer, cancer classification is performed from healthy DNA.

In particular, in methods herein described given the underlying evolution channel of the genome, approaches herein described capture information about the rate of generation of these mutations (the intrinsic mutation rate) from a single DNA, it might allow one to capture a signal about the propensity of the genome to incur these driver mutations. The mutation rates in tandem repeat regions are strong [10] and measurable [27]. In embodiments herein described one can estimate the evolutionary history of short tandem repeat regions or microsatellites and aggregate it to provide the genome's mutation profile which carries information about the number of duplications and point mutations required in the evolution of each tandem repeat region in the DNA. Using DNA derived from blood or healthy tissue of cancer patients from The Cancer Genome Atlas (TCGA) [32], one can estimate the mutation profiles for more than 5000 DNA samples on TCGA covering 14 different cancers including common cancers like lung, prostate, stomach, pancreas, skin, kidney, brain, etc. By successfully classifying different cancer-types based on the mutation profiles of the healthy genome, it is shown that these mutation profiles carry a cancer-type signal. By dividing this data into a training and a test set, one can build gradient boosting [33] based pairwise and multi classifiers that use mutation profiles as features to check if they carry any cancer-type signal [34, 35]. Based on these classifiers, one can generate cancer classification profiles which measured the propensity of an individual to each cancer type [34, 35]. As the cancer-type signal detection can be performed using genomes from healthy tissue, these mutation profiles could be useful in predicting future cancer risk and early cancer detection.

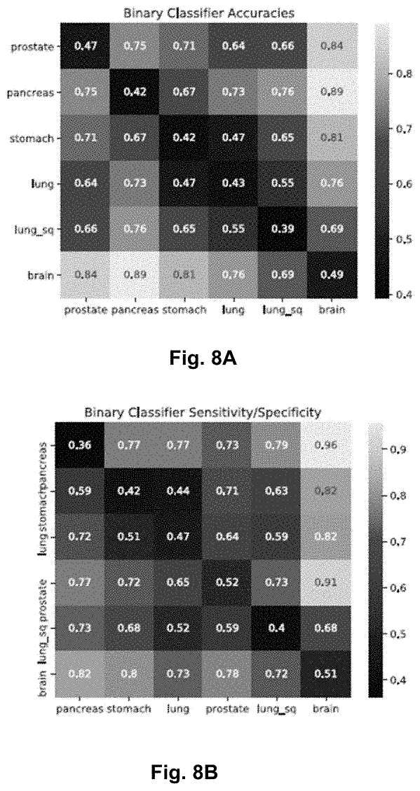

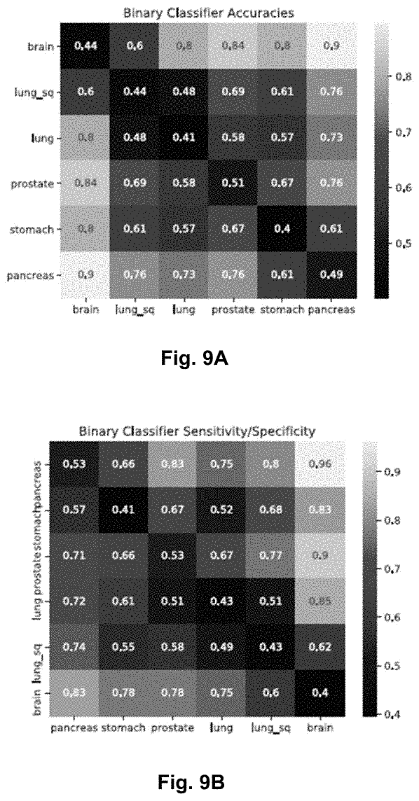

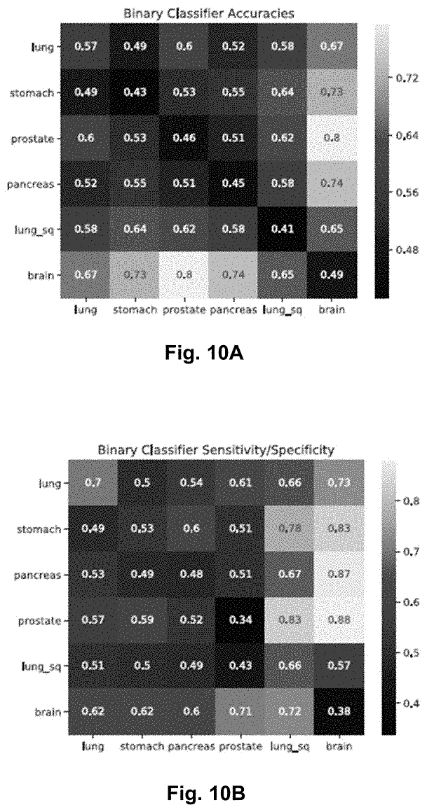

FIGS. 8A, 8B, 9A, 9B, 10A, and 10B show example accuracy (FIGS. 8A, 9A, and 10A) and sensitivity/specificity (FIGS. 8B, 9B, and 10B) matrices for pairwise binary classifiers trained on equal number of points from each listed cancer. It should be noted that the actual values presented for these figures might be suboptimal due to noise/bias introduced by having the samples having a mix of amplification/sequencing techniques performed on them. Results for samples from uniform amplification/sequencing is shown in FIGS. 16A, 16B, and 18A-19B. Pairwise classifiers in each figure were generated using a different set of features for training. In FIGS. 8A and 8B, mutation profiles are used as features to build pairwise classifiers. In FIGS. 9A and 9B, for each patient, a vector is obtained by taking the ratio of error number mᵢ and the copy number dᵢ for each repeated region i. These feature vectors are then used to obtain pairwise binary classifiers. In FIGS. 10A and 10B, the average value of the ratio mᵢ/dᵢ are used as features to create pairwise classifiers. Below are a few points useful in interpreting the numbers given by the matrices:

First, each cell in the seriation matrix represents the test accuracy of the binary pairwise classifiers. Each pairwise classifier between cancer X and cancer Y (for X≠Y) was constructed using 4-fold cross-validation with 100 patients of each cancer type. For example, the value in the cell corresponding to the row “stomach” and the column “prostate” signifies that an average of 71% of the people were correctly classified when 75 patients each for stomach and prostate cancer were used for training and 25 patients each for stomach and prostate cancer were used for testing in each of the 4 validation passes. The diagonal entries in the seriation matrix represent the average test accuracies using 4-fold cross validation when 50 patients of cancer X were labeled 0 and 50 patients of the same cancer X were labeled 1. As one can expect, the average test accuracy for such classifier should be around 50% (in some cases the average test accuracies observed along the diagonal are a bit lower or higher than 50% due to slight overfitting of the data).

An embodiment herein provides a method to identify a distance between different type of conditions, the method comprising building at least one classifier, wherein a first condition and the second condition are classified by the at least one classifier; determining an accuracy for the first condition classification against the second condition; and determining a distance based on the accuracy.

One can view these accuracies as distances, since similar cancers are harder to distinguish between. The order of the cancers in the display minimizes the distances between neighboring cancers, giving a likely one-dimensional projection of the features being learned by the classifiers. If the accuracies are provided from 0 to 1, then an accuracy close to 0.5 would be “near” (as in, difficult to distinguish between) and an accuracy close to 1 would be “far” (as in, easy to distinguish between). These distances can be used to group different conditions together as a single class by providing some threshold accuracy value (such as greater than 0.8) for conditions to be in the same class. These distances can also be used to infer a risk propensity from one condition to another. For example, if condition A and condition B have a condition distance of 0.9, then discovering that an individual is at risk for condition A means that one can infer that they are also at risk for condition B. Similarly, if a person has an ancestor (e.g. a parent) that had condition A, then they should be not only tested for condition A, but also for near-by condition B.

One embodiment for constructing a cancer risk profile for an individual uses the following steps:

Step 1: The mutation profile for the individual is first passed as an input to each of the N (e.g. 15) pairwise classifiers.

Step 2: Let a₁, a₂, a₃, a₄, a₅ and a₆ (etc.) be the number of classifiers that predicted prostate, lung, squamous cell lung, brain, pancreas and stomach (etc.) cancer respectively. Then, the cancer risk profile for the individual is given by the vector [a₁, a₂, a₃, a₄, a₅, a₆]. Each aᵢ, denotes the risk of having cancer i. Note that in this example, aᵢ≤5 and Σᵢ₌₁⁶ aᵢ=15.

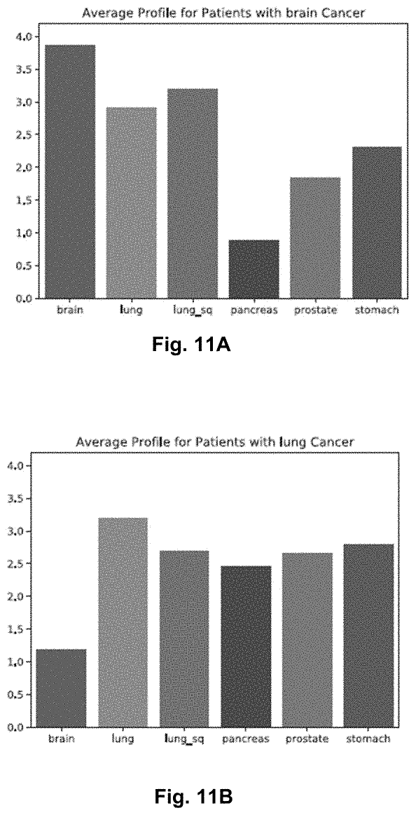

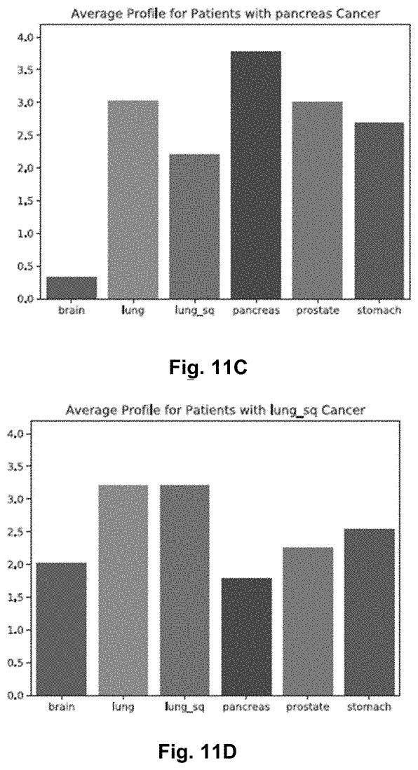

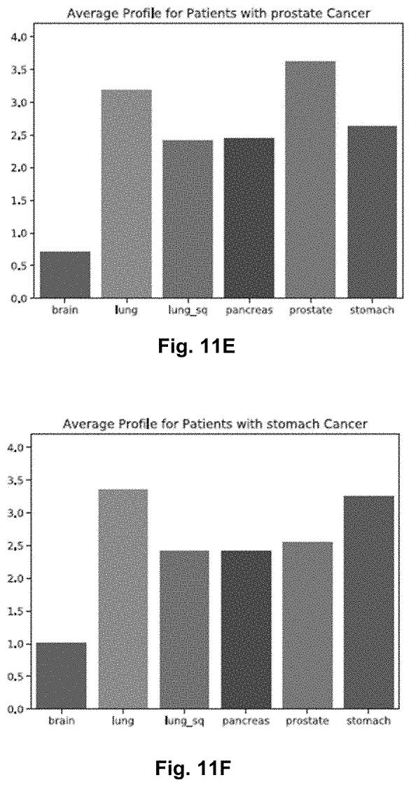

Applying this method of calculating cancer risk profile on healthy cell DNA of prostate, lung, squamous cell lung, brain, pancreas and stomach cancer patients provides FIGS. 11A-11F, showing the average values of risks associated with different cancers for patients with different reported cancers estimated by the algorithm. Generally, there is agreement, with the highest risk values corresponding to the actual cancer the patient was diagnosed with.

Measuring how often the reported cancer for the patient also is in the top 3 cancer risks in the cancer risk profile predicted by the classifier is shown in Table I. More sophisticated rank aggregation techniques mentioned in [18][35][36] and the references therein to build the cancer risk profile using these pairwise classifiers can also be used. Moreover, the pairwise classifiers can be based on hard decisions or soft decisions. Soft decisions provide that the confidence with which a classifier predicts a certain kind of cancer can be accounted for in predicting the cancer risk profile.

A soft decision refers to replacing the binary classifiers of the hard decision model with continuous classifiers, for example outputting a real number from 0 to 1 instead of only outputting a 0 or a 1. use these real number outputs as the confidence with which the classifier predicts a certain disease. These confidences will help us predict disease risk profile more accurately as they will reduce noise around the decisions made with pairwise classification where the risk of the two diseases does not differ by much. For example, a soft decision based pairwise classifier might predict 51% chances of prostate vs 49% chances of lung cancer, while on the other hand a binary classifier would just predict prostate cancer. In predicting the cancer risk profile in the latter case, the hard decision model gives a weight of 1 to prostate cancer, however in the soft decision-based approach, the weight given to prostate will be very small and will be almost close to 0 which is more accurate.

Table I: results for the percentage of patients for which their diagnosed cancer was within the top three cancers in their profiles from pairwise binary classifiers.

TABLE I

| Cancer | Accuracy in Top Three |
|---|---|
| Brain | 76 ± 0% |
| Lung | 73 ± 9% |
| Lung (squamous) | 68 ± 9% |
| Pancreas | 86 ± 2% |
| Prostate | 80 ± 8% |
| Stomach | 79 ± 10% |

### Method 2

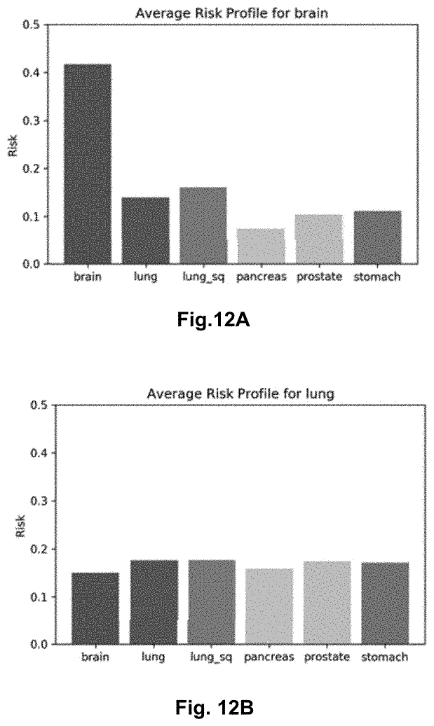

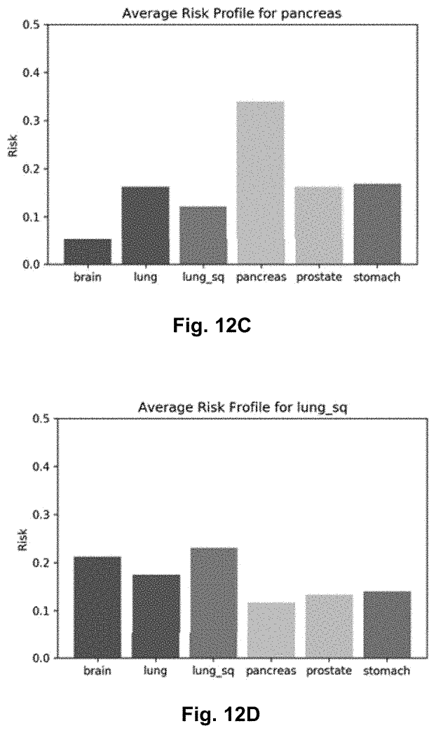

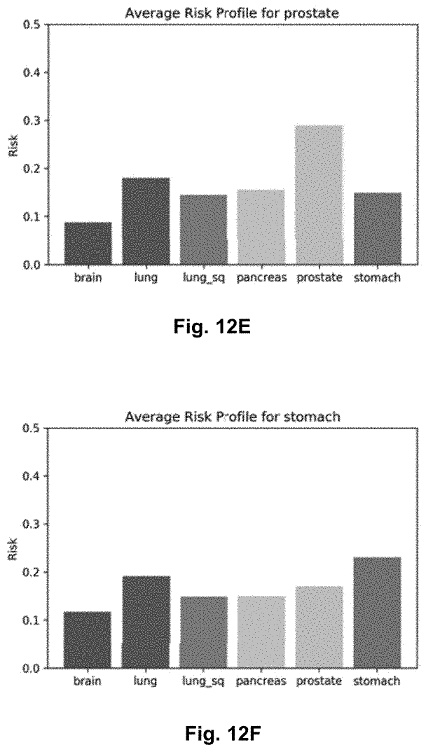

Another embodiment builds a multi-classifier which predicts probabilities representing the risk for each kind of cancer considered namely brain, squamous cell lung, lung, prostate, pancreas and stomach cancer. This was done using gradient boosting algorithm. The average risk profiles predicted by this classifier for patients of all the 6 cancer types in shown in FIGS. 12A-12F. Table II shows the times the reported cancer was also in the top 3 risks or probabilities in the cancer risk profile predicted by the classifier.

Table II: results for the percentage of patients for which their diagnosed cancer was within the top three cancers in their profiles using gradient boosting based multi-classifier.

TABLE II

| Cancer | Accuracy in Top Three |
|---|---|
| Brain | 79 ± 6% |
| Lung | 71 ± 12% |
| Lung (squamous) | 73 ± 5% |
| Pancreas | 83 ± 7% |
| Prostate | 70 ± 7% |
| Stomach | 70 ± 7% |

The prediction accuracies for the pairwise binary classifiers in FIGS. 8A-10B support the conjecture that healthy cell DNA carries signal about the cancer risk. For example, a prediction accuracy of 84% for the binary classifier between prostate and brain cancer in FIG. 8A suggests that with 84% accuracy, individuals with prostate and brain cancer risks can be differentiated by using the mutation profile of their healthy cell DNA. With these systems and methods, cancers can be differentiated based on healthy cell DNA. All the previous GWAS studies have focused on tumor cell DNA to do cancer detection. The probable reason previous GWAS studies focused on tumor cell DNA is because the tumor cell DNA carries somatic mutations and the healthy cell DNA only have germline mutations. However, the approach of capturing evolution information using mutation profile is focused on inferring the tendency of DNA to develop somatic mutations in the future. Thereby, using healthy cell DNA, one can infer some information about this tendency to undergo many stem cell divisions and thereby generate somatic mutations.

Near 50% prediction accuracies in the diagonals on FIGS. 8A-10B also suggest that the mutation profile and the ratio of error number and copy number (mᵢ/dᵢ) is very similar in the healthy cell DNA for individuals with the same cancer type, showing that mutation profile and mᵢ/dᵢ captures the evolution information well enough.

Near 50% accuracies for pairwise classifiers between lung and stomach cancer or lung and squamous cell lung cancer also verify the findings in [37], that lung and stomach cancer are governed more by environmental factors and not by evolutionary factors, hence the evolutionary information captured by mutation profile is not distinguishable.

Further, the pairwise classifiers perform better prediction in general when mutation profiles of healthy cell DNA are used as features (see FIGS. 8A and 8B) compared to using the ratio of error and copy number (see FIGS. 9A and 9B) or the average value of this ratio (see FIGS. 10A and 10B) which means mutation profile captures more evolutionary information than these ratios.

The average risk profiles plot shown in FIGS. 11A-F and 12A-F for patients with different kinds of cancer is also in consistence with the conjecture that healthy cell DNA carries useful evolutionary information that can be used for creating a disease risk profile. As it can be seen, for brain, pancreas and prostate cancer patients, the rank aggregator classifier (see FIGS. 11A-F) also shows highest risks for the corresponding cancers. Further lung, squamous cell lung and stomach cancer are caused by environmental factors, therefore for the patients with these cancers, the average risk profiles share lesser variance for the risks of lung, squamous cell lung and stomach cancer. A similar trend is seen in the average risk profiles estimated by the multi-classifier in FIGS. 12A-F with brain, pancreas and prostate cancer clearly showing the highest risk for the patients reported with the respective cancer. However, in the case of lung, squamous cell lung and stomach cancer patients, the top 2 average risk values are relatively closer as these cancers are caused majorly due to environmental factors.

Further, the highest risk value for environmental mutation cancers is smaller than the highest risk values for random mutation cancers strengthening the conjecture that the healthy cell DNA carries information about the random mutation related cancers.

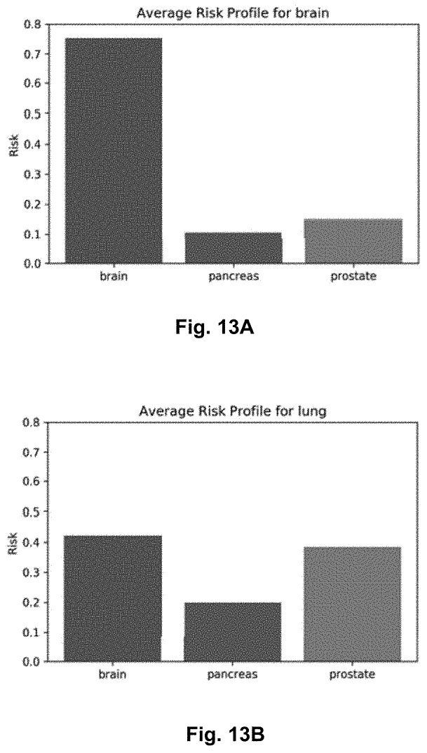

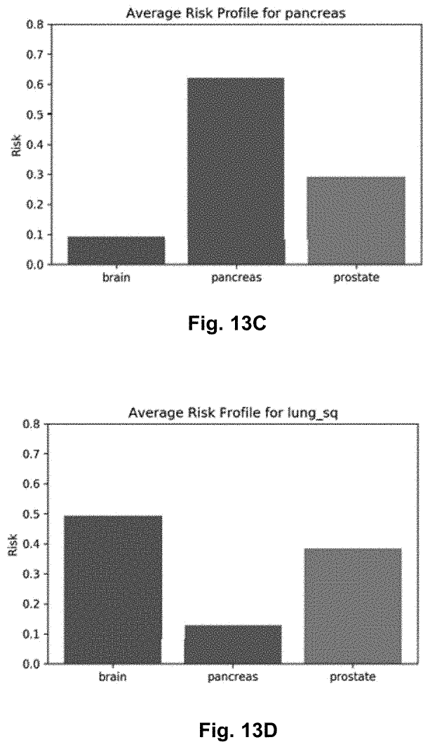

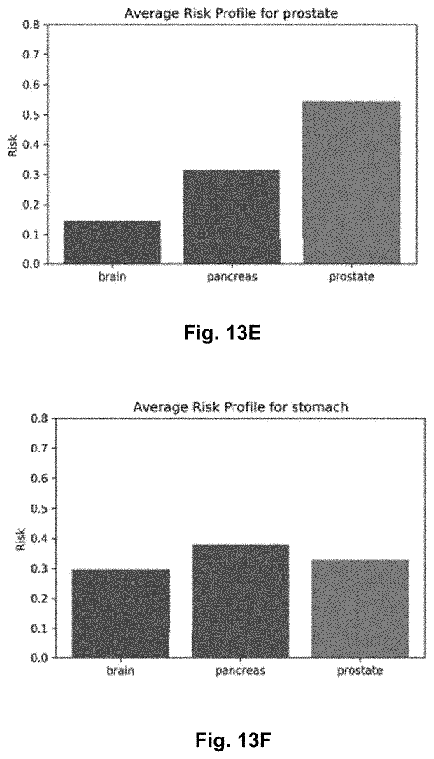

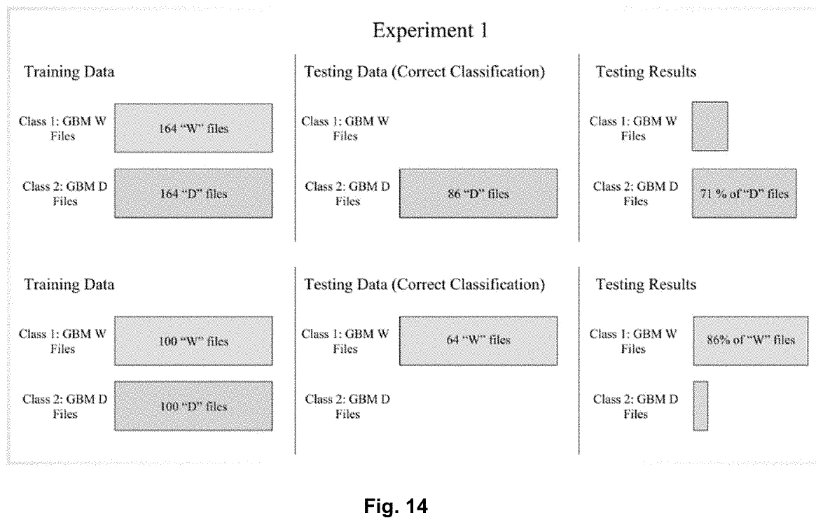

Prostate, pancreas and brain cancer have been shown to be primarily caused by random mutations in [37]. FIGS. 13A-13F show the average risk profiles predicted using the gradient boosting based multi-classification with training done using prostate, pancreas and brain cancers. The left column in these figures show that the highest risk predicted by the classifier corresponds to the reported cancer for the patient. Further, the right column here shows the risk of these random mutation related cancers on the patients reported with lung, squamous cell lung and stomach cancer respectively.

Tables I and II also solidify the applicability of the disease risk profile estimation algorithms mentioned in Method 1 and Method 2 respectively. For example, in about 76% cases (see Table I), the risk profile estimated by the rank aggregation algorithm mentioned in Method 1 predicts brain cancer in the top 3 risks for people with brain cancer using their healthy cell DNA. Results of similar nature are observed by the application of Method 2 (see Table II).

In general, the findings here show for the first time that the mutation profile extracted from the healthy cell DNA carries information about the risk of getting cancer which can be used to develop inexpensive computational clinical tests that can be used for early cancer detection or to estimate cancer risks in healthy individuals and enable targeted protocols for screening and early detection.

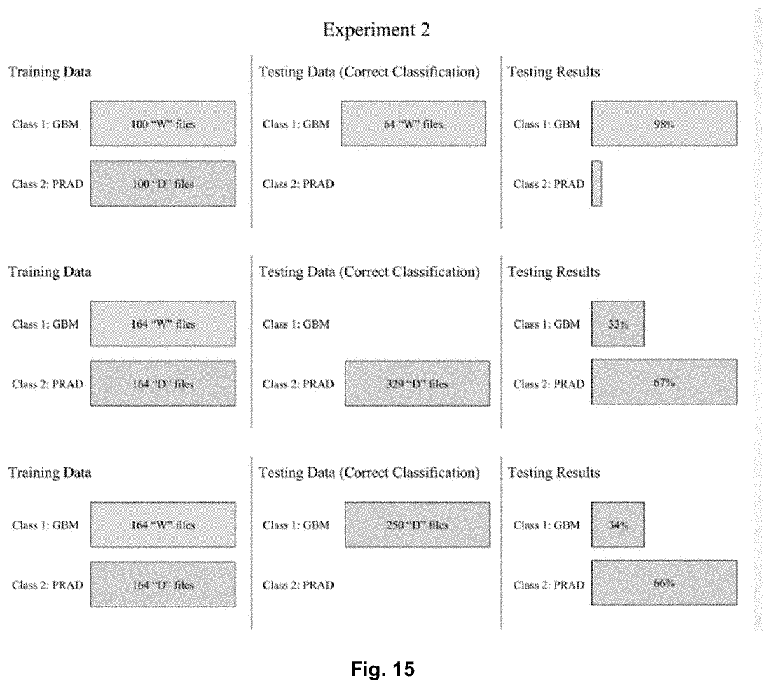

FIG. 14 shows an example of training and testing a machine learning classifier. This figure represents the issue of data bias that can be present in the analysis of tandem repeat regions when samples from different amplification techniques are used. “D” and “W” represents unamplified and amplified samples respectively. Here, the placement of bars represent the labeling of the classes. The first column shows the data that our classifier was trained on. The second column shows where a perfect classifier would put the data, and the third column shows how our classifier labeled that data. Here it is seen that, within the cancer class of GBM, one can train a fairly accurate classifier for “D” and “W” files.

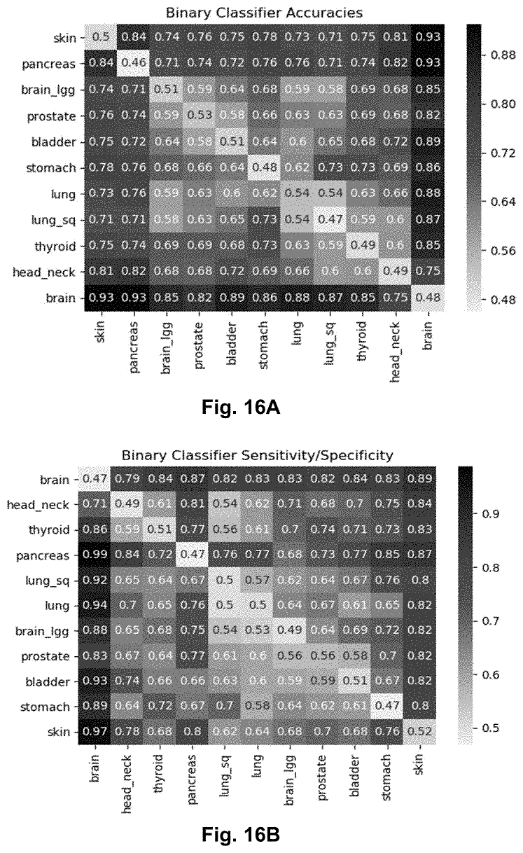

FIG. 15 shows a further example of training and testing a machine learning classifier. They represent how amplification noise can interfere with the cancer signal. GBM represents Giloblastoma and PRAD represents Prostate Adenocarcinoma. As in FIG. 14, the placement of bars represent the labeling of the classes, which this time is separated by cancer type (GBM vs PRAD). Here, a classifier trained on cancers which differ in their file type appears to be successful in the first two test sets i.e., GBM “W” files and PRAD “D” files. The testing results for the third test set GBM “D” files, however, shows that the classification of GBM “D” files is very similar to that of PRAD “D” files. Hence, the machine learning algorithm has mistaken the D/W signal for the cancer-type.

FIGS. 16A and 16B show example accuracy (FIG. 16A) and sensitivity/specificity (FIG. 16B) matrices for pairwise binary classifiers built by using 3843 blood-derived normal DNA samples (unamplified) covering 11 different cancer types. For these examples, the cancer types are TCGA-SKCM (skin), PAAD (pancreas), STAD (stomach), BLCA (bladder), PRAD (prostate), LGG (brain_lgg), LUAD (lung), THCA (thyroid), LUSC (lung_sq), HNSC (head_neck), GBM (brain). Each cell in the accuracy seriation matrix represents the average validation accuracy of the binary pairwise classifiers. Each pairwise classifier between cancer X and cancer Y (for X≠Y) was constructed using 4-fold cross-validation with patients of each cancer type. These accuracies can be interpreted as distances—the higher the accuracy, the more distinguishable (i.e. different) the cancers are, and so the further apart the cancer types are from each other. For example, in FIG. 16A, the darker the matrix cell, the farther apart are the cancers being compared. The darker rows corresponding to brain, skin and pancreas are indicative of the presence of cancer-type signal in the blood-derived normal (healthy) DNA of cancer patients. Note that while FIGS. 16A and 16B involve results for unamplified samples, FIGS. 8-13 involve results for samples of mixed amplification (see, e.g., FIGS. 14 and 15) to show the effect of amplification bias.

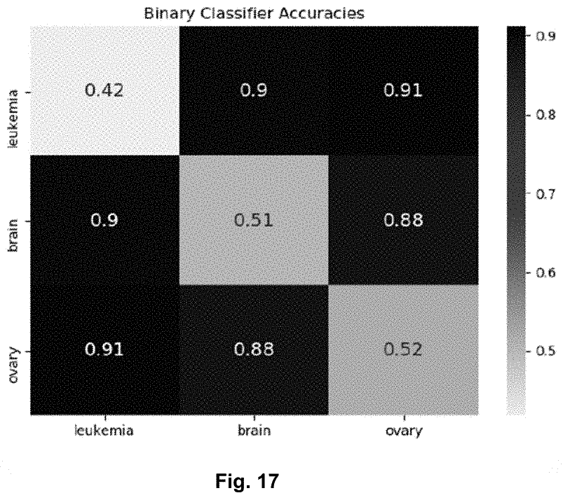

FIG. 17 shows example accuracies for a binary classifier for leukemia, brain, and ovary cancer risks when only amplified samples are used. One can see that the cancer signal remains even with amplification.

The diagonal entries in the seriation matrix represent the accuracies when half of the patients of cancer X were labeled 0 and half of the patients of the same cancer X were labeled 1. As one can expect, the average test accuracy for such classifier should be around 50%. The value in the cell corresponding to the row “pancreas” and the column “prostate” signifies that an average of 74% of the people were correctly classified in each validation pass. The matrix on the right, contains the sensitivity/specificity values. Each cell in the sensitivity/specificity seriation matrix represents the sensitivity value when the row cancer is considered positive and the column cancer is considered negative. It can also be regarded as specificity when the row cancer is considered negative and the column cancer is considered positive.

Sensitivity is defined as TP/(TP+FN) and specificity is defined as TN/(TN+FP), where TP=True Positive, FP=False Positive, TN=True Negative, FN=False Negative. A value of 0.77 in the row “prostate” and the column “pancreas” means that 77% of the prostate patients in the test set were truly classified as prostate type (sensitivity when prostate is considered positive). A value of 0.73 in the row “pancreas” and the column “prostate” means that 73% of the pancreas patients in the test set were truly classified as pancreas type (specificity when prostate is considered positive).

The seriation ordering can be obtained by solving TSP (., the Travelling Salesman Problem) exhaustively, thereby minimizing the distances between neighboring cancers. Cancers with risk factors that emit different mutation profiles are easier to distinguish, resulting in more accurate classifiers. Hence, accuracy gives a notion of distance on the scale of 50% (close, indistinguishable) to 100% (far, different).

Table III shows (A) Number of unamplified healthy samples used for each cancer type in the study showing the number of blood derived normal and solid tissue normal samples. In total, the number of blood derived healthy samples are 3874 and the tissue derived healthy samples are 687. (B) Number of amplified healthy samples used for each cancer type in the study showing the number of blood derived normal and solid tissue normal samples. In total, the number of blood derived healthy samples are 331 and the tissue derived healthy samples are 194.

TABLE III

| | | |
|---|---|---|
| | Blood Derived | Solid Tissue |
| Cancer | Normal | Normal |

A: Unamplified Samples

| | | |
|---|---|---|
| SKCM | 344 | 0 |
| PAAD | 153 | 31 |
| STAD | 396 | 49 |
| BLCA | 393 | 20 |
| PRAD | 440 | 56 |
| LGG | 513 | 0 |
| LUAD | 411 | 102 |
| THCA | 432 | 68 |
| LUSC | 316 | 180 |
| HNSC | 190 | 0 |
| GBM | 255 | 2 |
| KIRC | 31 | 179 |

B: Amplified Samples

| | | |
|---|---|---|
| GBM | 171 | 0 |
| LAML | 0 | 135 |
| OV | 160 | 59 |

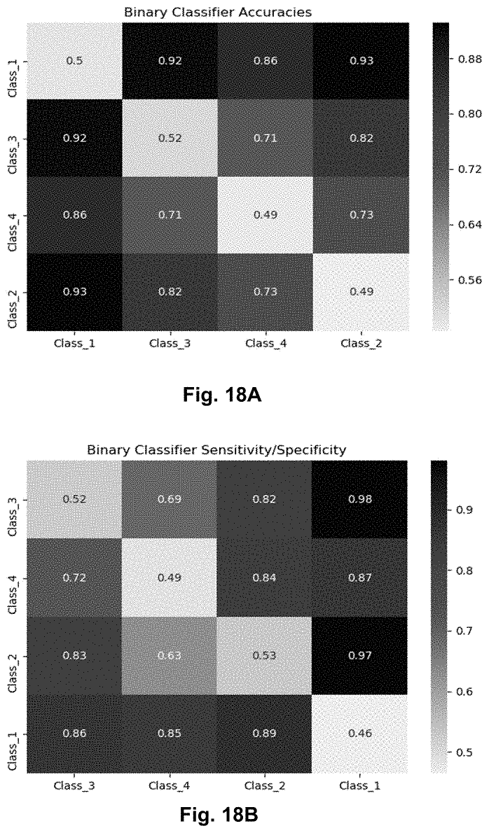

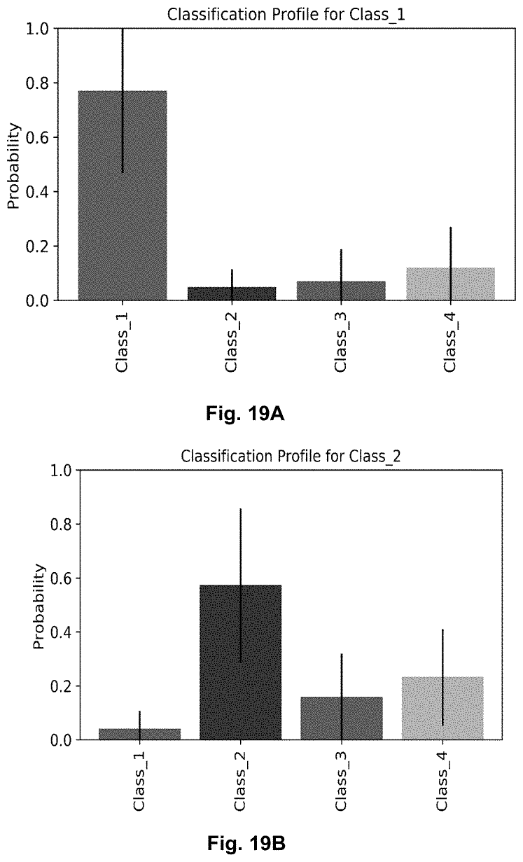

FIGS. 18A and 18B show example 4-fold validation accuracy (FIG. 18A) and sensitivity/specificity (FIG. 18B) for the four main clusters of cancers in FIGS. 17A and 17B generated using 3843 blood-derived normal samples. Class 1=(brain), Class 2=(skin), Class 3=(pancreas), Class 4=(stomach, bladder, prostate, brain_lgg, lung, thyroid, lung_sq, head_neck).

FIGS. 19A and 19B show example mean and standard deviations for the cancer classification profiles of individuals in Class 1 (FIG. 19A) and Class 2 (FIG. 19B). To generate these profiles, trained multi-classifier is trained on all four classes of cancers using gradient boosting. This multi-classifier can then be used to obtain cancer classification profiles for a different set of individuals reported the average results for each cancer class. Class 1 individuals show a high probability for Class 1 cancers. Class 2 individuals also show a higher probability for Class 2 cancers, but with a slightly weaker signal.

In general, the findings here show for the first time that the mutation profile extracted from DNA of any cell, including “healthy” cells, carries information about the risk of getting cancer which can be used to develop inexpensive computational clinical tests that can be used for early cancer detection or to estimate cancer risks in healthy individuals and enable targeted protocols for screening and early detection.

In some embodiments, methods and systems described herein can be performed based on an age based analysis.

For example, cancer is a disease that can be modeled as a stochastic process with some individuals at higher risk than others. One can model cancer's occurrence as a Poisson distribution with parameter λ. The distribution of the time in between occurrences of a Poisson process is exponential. Thus, if the random variable t represents when an individual with parameter λ gets cancer, then:

t exp(λ)

This distribution's probability density function is λe^{-λt}. Thus, the likelihood that an individual gets cancer at age t is given by:

Λₜ⁺(λ)=∫₀^{t}λe^{-λt}dt=1−e^{-λt}

One can also use this distribution to find the likelihood in the negative case. The likelihood that an individual has not gotten cancer by time t is given by:

Λₜ⁻(λ)=1−∫₀^{t}λe^{-λt}dt=e^{-λt}

To find the function λ(m, d) such that the likelihood of the data is maximized:

$${argmax}_{λ ( m , d )} {\prod }_{( t , m , d ) ∈ has cancer} {Λ}_{t}^{+} ( λ ) \times {\prod }_{t ∈ healthy} {Λ}_{t}^{-} ( λ ) = {argmax}_{λ ( m , d )} {\prod }_{( t , m , d ) ∈ cancer} 1 - {e}^{- λ ( m , d ) t} \times {\prod }_{t ∈ healthy} {e}^{- λ ( m , d ) t} = {argmax}_{λ ( m , d )} exp [ {\prod }_{( t , m , d ) ∈ cancer} log ( 1 - {e}^{- λ ( m , d ) t} ) ] exp [ - {\sum }_{( t , m , d ) ∈ healthy} λ ( m , d ) t ]$$

Taking the negative logarithm allows us to express this problem as a minimization of loss functions for each point.

Loss⁺(v,t)=−log(1−e^{-λ(v)t})

Loss⁻(v,t)=λ(v)t

If (m, d)=v and assume the model for λ(m, d)=c^{T}v, one can see that the gradients for stochastic gradient descent are

$$∇ {Loss}^{+} ( v , t ) = - v \frac{{te}^{- λ ( v ) t}}{1 - {e}^{- λ ( v ) t}}$$

$$∇ {Loss}^{-} ( v , t ) = vt$$

### More Datasets

Other healthy cell DNA data can be collected for more cancer patients from TCGA database with cancer already covered herein and for other cancers like neck, cervical, breast, colon, rectum, leukemia to name a few. The DNA data of healthy people with no history or evidence of cancer can also be collected.

As mentioned earlier, these ideas are not specific to estimating cancer risk propensity only. It is fairly general and can be used for any mutation-based disease like Alzheimers, Parkinson's, Autoimmune diseases, etc. to predict a disease risk propensity.

### Multi-Classification

As mentioned above, a multi-classifier, optionally based on a gradient boosting algorithm, can be used which predicts the probability of multiple cancers at once.

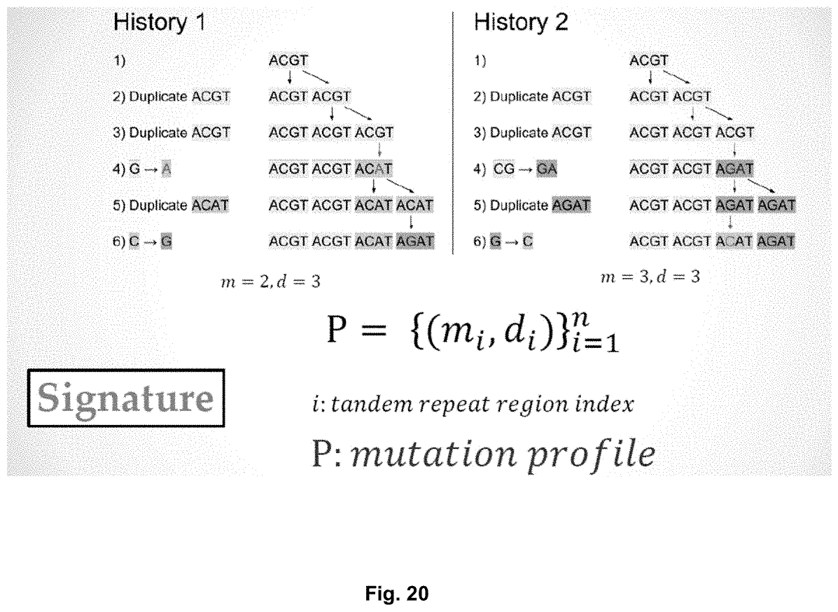

FIG. 20 shows an example of the mutation profile at index i for two histories. For History 1, the mutation index (m, d) would be (2, 3), representing two error (mutation) events (at steps 4 and 6 in the history) and three repeat events (at steps 2, 3, and 5). For History 2, the (m,d) at that index would be (3, 3) for three error (steps 4 having two errors—C->G and G->A, plus step 6) and three duplication (steps 2, 3, and 5). Although the substitution of CG->GA might have been a single event, it is considered two substitution errors for the purposes of the mutation profile.

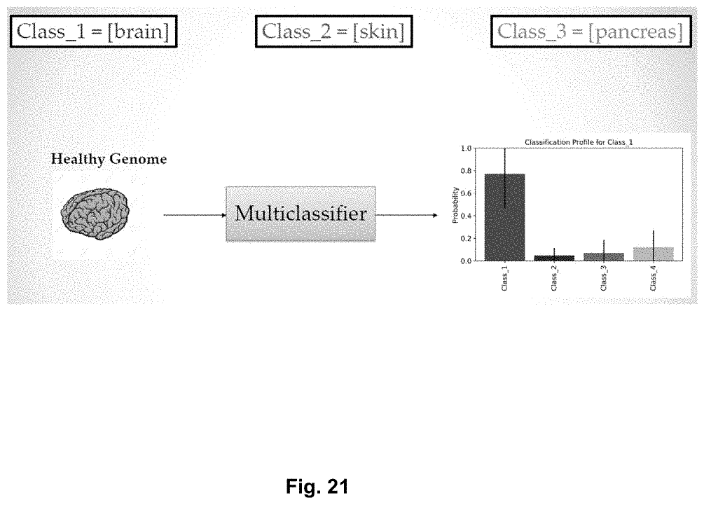

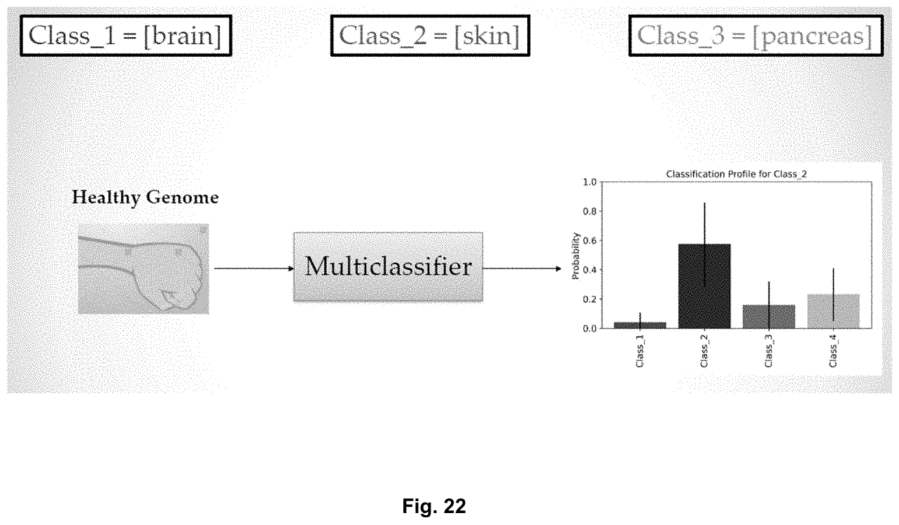

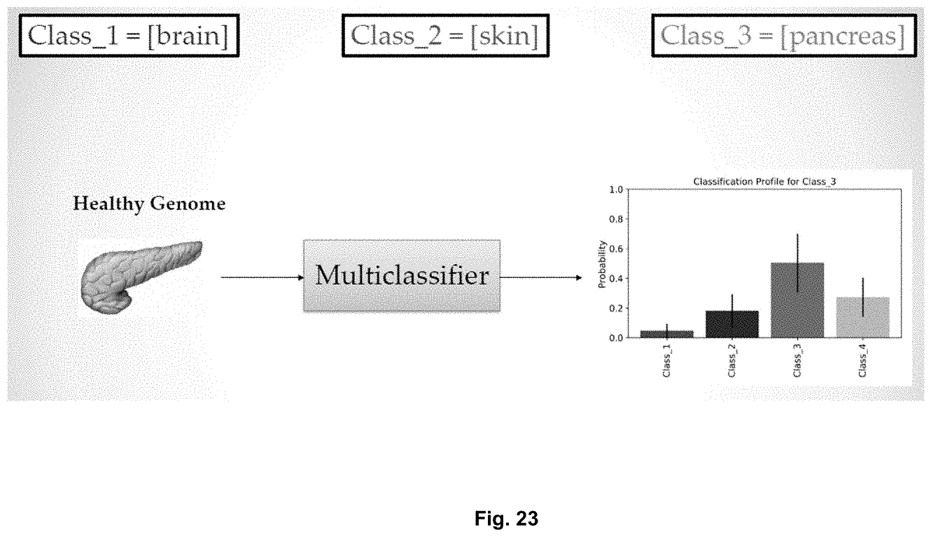

FIGS. 21, 22, and 23 show example risk propensities derived from a multi-classifier for brain, skin, pancreatic, and “other” cancer. FIG. 21 shows an identification of high brain cancer risk, FIG. 22 shows an identification of high skin cancer risk, and FIG. 23 shows an identification of high pancreatic cancer risk. Class 4 represents the other cancers in the multi-classifier (for example, brain (lower grade giloma), prostate, lung, squamous cell lung, head and neck, stomach, bladder, thyroid).

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure. Those skilled in the art will recognize how to adapt the features of the exemplified methods and related systems, hardware and compositions to various embodiments and scope of the claims.

All patents and publications mentioned in the specification are indicative of the levels of skill of those skilled in the art to which the disclosure pertains.

The entire disclosure of each document cited (including webpages patents, patent applications, journal articles, abstracts, laboratory manuals, books, or other disclosures) in the Field, Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

Definitions that are expressly set forth in each or any claim specifically or by way of example herein, for terms contained in relation to features of such claims are intended to govern the meaning of such terms. The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, no limitation, element, property, feature, or attribute that is not expressly recited in a claim should limit the scope of such claim in any way. The specification and drawings are, accordingly, to be regarded in an illustrative rather than a restrictive sense Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

### REFERENCES

[1] E. S Lander, L. M Linton, B. Birren, C. Nusbaum, M. C Zody, Jennifer Baldwin, Keri Devon, Ken Dewar, M. Doyle, William FitzHugh, and others. “Initial Sequencing and Analysis of the Human Genome”. In: Nature 409.6822 (2001), pp. 860-921.
[2] N. I. Mundy and A. J Helbig. “Origin and Evolution of Tandem Repeats in the Mitochondrial DNA Control Region of Shrikes (Lanius Spp.)” In: Journal of Molecular Evolution 59.2 (2004), pp. 250-257.
[3] C. Tomasetti, L. Li, and B. Vogelstein, “Stem cell divisions, somatic mutations, cancer etiology, and cancer prevention,” Science no. 355, vol. 6331, pp. 1330-1334, 2017.
[4] R. J. Hause, C. C. Pritchard, J. Shendure, and S. J. Salipante, “Classification and characterization of microsatellite instability across 18 cancer types,” Nature medicine, vol. 22, no. 11, pp. 1342-1355, 2016.
[5] L. J. McIver, N. C. Fonville, E. Karunasena, and H. R. Garner, “Microsatellite genotyping reveals a signature in breast cancer exomes,” Breast cancer research and treatment, vol. 145, no. 3, pp. 791-798, 2014.
[6] T. B. Sonay, M. Koletou, and A. Wagner, “A survey of tandem repeat instabilities and associated gene expression changes in 35 colorectal cancers,” BMC genomics, vol. 16, no. 1 pp. 702-713, 2015.
[7] L. Wang, J. C. Soria, Y. S. Chang, H. Y. Lee, Q. Wei, and L. Mao, “Association of a functional tandem repeats in the downstream of human telomerase gene and lung cancer,” Oncogene, vol. 22, no. 46 pp. 7123-7129, 2003.
[8] K. Usdin. “The Biological Effects of Simple Tandem Repeats: Lessons from the Repeat Expansion Diseases”. In: Genome research 18.7 (2008), pp. 1011-1019.
[9] J. W. Fondon and Harold R. Garner. “Molecular Origins of Rapid and Continuous Morphological Evolution”. In: Proceedings of the National Academy of Sciences 101.52 (2004), pp. 18058-18063. doi: 10.1073/pnas.0408118101.
[10] J. X. Sun, A. Helgason, G. Masson, S. S. Ebenesersdóttir, H. Li, S. Mallick, S. Gnerre, N. Patterson, A. Kong, D. Reich, and K. Stefansson. “A Direct Characterization of Human Mutation Based on Microsatellites”. en. In: Nature Genetics 44.10 (October 2012), pp. 1161-1165. issn: 1061-4036. doi:10.1038/ng.2398.
[11] G Levinson and G A Gutman. “Slipped-Strand Mispairing: A Major Mechanism for DNA Sequence Evolution.” In: Molecular Biology and Evolution 4.3 (1987), pp. 203-221.
[12] C. Schlötterer. “Evolutionary Dynamics of Microsatellite DNA”. en. In: Chromosoma 109.6 (September 2000), pp. 365-371. issn: 0009-5915, 1432-0886. doi: 10.1007/s004120000089.
[13] G. Benson, “Tandem repeats finder: a program to analyze DNA sequences,” Nucleic acids research, vol. 27, no. 2, pp. 573-581, 1999.
[14] M. Tang, M. Waterman, and S. Yooseph, “Zinc finger gene clusters and tandem gene duplication,” Journal of Computational Biology, vol. 9, no. 2, pp. 429-446, 2002.
[15] T. Warnow. Computational Phylogenetics: An Introduction to Designing Methods for Phylogeny Estimation. Cambridge University Press, 2017. doi:10.1017/9781316882313.
[16] Smith, T. F. & Waterman, M. S., “Identification of Common Molecular Subsequences” Journal of Molecular Biology 147(1), 195-197 (1981)
[17] Benson, G., “Tandem repeats finder: a program to analyze DNA sequences”, Nucleic Acids Research 27(2), pp. 573-580 (1999)
[18] C. E. Shannon. “A Mathematical Theory of Communication”. In: The Bell System Technical Journal 27.3 (July 1948), pp. 379-423. issn: 0005-8580. doi: 10.1002/j.1538-7305.1948.tb01338.x.
[19] B. W. Stewart and C. P. Wild. World Cancer Report. Lyon, France: IARC, 2014.
[20] TCGA data portal: gdc-portal.nci.nih.gov.
[21] D. J. Burgess, “Human genetics: Somatic mutations linked to future disease risk,” Nature Reviews Genetics, p. 69, 2015.
[22] A. Poduri, G. D. Evrony, X. Cai, C. A. Walsh, “Somatic mutation, genomic variation, and neurological disease,” Science, vol. 341, no. 6141, 1237758, 2013.
[23] K. A. Ross, “Coherent somatic mutation in autoimmune disease,” PLOS One, vol. 9, no. 7, e101093, 2014.
[24] Ellrott, K., Bailey, M. H., Saksena, G., Covington, K. R., Kandoth, C., Stewart, C., Hess, J., Ma, S., Chiotti, K. E., McLellan, M., et al. (2018). Scalable Open Science Approach for Mutation Calling of Tumor Exomes Using Multiple Genomic Pipelines. Cell Syst. 6, 271-281.e7.
[25] Cibulskis, K., McKenna, A., Fennell, T., Banks, E., DePristo, M., and Getz, G. (2011). ContEst: estimating cross-contamination of human samples in next-generation sequencing data. Bioinformatics 27, 2601-2602.
[26] Costello, M., Pugh, T. J., Fennell, T. J., Stewart, C., Lichtenstein, L., Meldrim, J. C., Fostel, J. L., Friedrich, D. C., Perrin, D., Dionne, D., et al. (2013). Discovery and characterization of artifactual mutations in deep coverage targeted capture sequencing data due to oxidative DNA damage during sample preparation. Nucleic Acids Res. 41, e67-e67.
[27] M. Tang, M. Waterman, and S. Yooseph. “Zinc Finger Gene Clusters and Tandem Gene Duplication”. In: Proceedings of the Fifth Annual International Conference on Computational Biology. RECOMB '01. Montreal, Quebec, Canada: ACM, 2001, pp. 297-304. isbn: 1-58113-353-7. doi:10.1145/369133.369241. url: doi.acm.org/10.1145/369133.369241.
[28] F. Farnoud, M. Schwartz, and J. Bruck. “Estimation of duplication history under a stochastic model for tandem repeats”. In: BMC Bioinformatics 20.1 (2019), p. 64. issn: 1471-2105. doi: 10.1186/s12859-019-2603-1. url: doi.org/10.1186/s12859-019-2603-1.
[29] N. Ailon, “An active learning algorithm for ranking from pairwise preferences with an almost optimal query complexity,” Journal of Machine Learning Research, vol. 13, pp. 137-164, 2012.
[30] N. B. Shah, M. Wainwright, “Simple, Robust and Optimal Ranking from Pairwise Comparisons,”arXiv:1512.08949v2 [cs.LG], 2016.
[31] R. Heckel, M. Simchowitz, K. Ramchandran, M. J. Wainwright, “Approximate Ranking from Pairwise Comparisons,” arXiv:1801.01253 [cs.LG], 2018.
[32] National Cancer Institute. About the Data NCI Genomic Data Commons. url: gdc.cancer.gov/about-data.
[33] L. Mason, J. Baxter, P. Bartlett, and M. Frean. “Boosting Algorithms As Gradient Descent”. In: Proceedings of the 12th International Conference on Neural Information Processing Systems. NIPS'99. Denver, Colo.: MIT Press, 1999, pp. 512-518. url: dl.acm.org/citation.cfm?id=3009657.3009730.
[34] S. Jain, B. Mazaheri, N. Raviv, and J. Bruck. “Cancer Classification from Healthy DNA using Machine Learning”. In: bioRxiv (2019). doi: 10.1101/517839.eprint: www.biorxiv. org/content/early/2019/01/11/517839.full.pdf. url: www.biorxiv.org/content/early/2019/01/11/517839.
[35] S. Ohno. Evolution by Gene Duplication. Springer-Verlag, 1970.
[36] C. McIntosh and S. D. Wilton. “Polyglutamine ataxias: From Clinical and Molecular Features to Current Therapeutic Strategies”. In: 2017.
[37] K. A. Schouhamer Immink and P. H. Siegel. “Codes for Mass Data Storage Systems (Second)”. In: 2004. Published in: IEEE Transactions on Information Theory (Volume: 52, Issue: 12, pp 5614-5616, December 2006)

1. A mutation profile of a cell of an individual, comprising:
a set of genome values representing history of repeat regions of at least a portion of the genome of the individual,
each genome value being numerically characterized by a value indicative of
i) a first number being representative of an error number of a repeat region of the repeat regions, and
ii) a second number being representative of a copy number of the repeat region,
the mutation profile indicative of development and diversification of the genome of the individual in time.

2. The mutation profile of claim 1, wherein the value is a multi-dimensional index comprising the first number and the second number.

3. The mutation profile of claim 1, wherein the value is a ratio between the first number and the second number or vice versa.

4. The mutation profile of claim 1, wherein the repeat regions are one or more of interspersed repeat regions, tandem type repeat regions, nested tandem repeat, regions, direct repeats, and inversed repeats.

5. The mutation profile of claim 1, wherein errors comprise nucleotide substitutions, deletions and/or insertions.

6. A non-transitory computer-readable medium comprising a training set for a learning algorithm, the training set comprising a plurality of mutation profiles according to claim 1.

7. A method for building a mutation profile for an individual, comprising:
obtaining a DNA sequence from the individual;
finding at least one repeat region in the DNA sequence;
evaluating a consensus pattern for each of the at least one repeat region;
determining a plurality of mutation histories for each of the at least one repeat region, each mutation history having a consensus pattern;
determining estimated histories for each of the plurality of mutation histories for each consensus pattern; and
building a mutation profile based on the estimated histories of the plurality of mutation histories for each consensus pattern.

8. The method of claim 7, wherein each of the estimated histories is a mutation history that has a least cost among a corresponding plurality of mutation histories.

9. The method of claim 7, wherein the mutation profile comprises a mutation index which comprises a copy number and an error number.

10. The method of claim 7, further comprising: compiling multiple mutation profiles from a plurality of individuals with a shared condition and using the mutation profiles to train a machine learning classifier for the shared condition.

11. The method of claim 10, further comprising: determining a new mutation profile from a target individual; and determining a disease risk propensity for the target individual by applying the machine learning classifier to the new mutation profile.

12. A method for determining a condition risk propensity for a target condition in an individual, the method comprising:
determining a first set of mutation profiles for a population of individuals with the target condition, each mutation profile of the first set of mutation profiles being the mutation profile of claim 1 for each corresponding individual of the population of individuals with the target condition;
determining a second set of mutation profiles for a population of individuals not having the condition, each mutation profile of the second set of mutation profiles being the mutation profile of claim 1 for each corresponding individual of the population of individuals not having the target condition;
training a classifier using the first set of mutation profiles and the second set of mutation profiles; and
running the classifier on a mutation profile of the individual such that a risk propensity for the target condition is generated the mutation profile of the individual being the mutation profile of claim 1 for the individual.

13. The method of claim 12, wherein the population of individuals not having the condition have a second condition different from the condition, and the risk propensity compares risk of the condition with risk of the second condition.

14. The method of claim 12 further comprising combining the risk propensity with other risk propensities to create a multiple condition risk propensity.

15. A method for determining a condition risk propensity for a plurality of target conditions in an individual, the method comprising:
determining a plurality of sets of mutation profiles for a plurality of populations, each of the plurality of populations having a corresponding target condition unique to that population, each mutation profile of the plurality of sets of mutation profiles being the mutation profile of claim 1 for an individual of the plurality of population;
training a classifier using the plurality of sets of mutation profiles, classifying by condition; and
running the classifier on a mutation profile of the individual such that a risk propensity is generated for the plurality of target conditions, the mutation profile of the individual being the mutation profile of claim 1 for the individual.

16. A method to predict a condition risk propensity of an occurrence of a target condition in an individual, the target condition associated with genetic factors, the method comprising:
detecting, in a cell of the individual, the mutation profile of claim 1, the detected mutation profile indicative of development and diversification of the genome of the individual in time; and
comparing the detected mutation profile with a reference mutation profile associated with the condition to provide the condition risk propensity for the individual.

17. The method of claim 16, wherein
the reference mutation profile comprises a first set of mutation profiles for a population of individuals with the condition and a second set of mutation profiles for a population of individuals not having the condition; and wherein
the comparing is performed by the method of claim 12.

18. The method of claim 16, wherein
the reference mutation profile comprises a plurality of sets of mutation profiles for a plurality of populations, each of the plurality of populations having a corresponding condition unique to that population; and wherein
the comparing is performed by the method of claim 15.

19. A labeled human genome component, comprising
at least a portion of a genome of an individual, in combination with the mutation profile of claim 1.

20. The labeled human genome component of claim 19, said at least a portion of the genome of the individual being a polynucleotide.

21. The labeled human genome component of claim 19, said at least a portion of the genome of the individual being a representation of said human genome.

22. A method to identify a distance between different type of conditions, the method comprising building at least one classifier, wherein a first condition and a second condition are classified by the at least one classifier; determining a classification accuracy for the first condition against the second condition; and determining a condition distance based on the classification accuracy.
